(19) United States
(12) Patent Application Publication
(10) Pub. No.: US 2026/0199548 A1
(43) Pub. Date: Jul. 16, 2026

(54) BIOCOMPATIBLE HYDROGEL COMPOSITION COMPRISING MESOPOROUS SILICA FOR DRUG DELIVERY AND USE THEREOF

(71) Applicant: CEN CO., LTD., Miryang-si (KR)

(72) Inventors: Sang Cheol HAN, Miryang-si (KR); Sang Mo KWON, Yangsan-si (KR); Woong Bi JANG, Yangsan-si (KR); Yong Il SHIN, Yangsan-si (KR)

(21) Appl. No.: 19/065,079

(22) Filed: Feb. 27, 2025

(30) Foreign Application Priority Data
Jan. 16, 2025 (KR) 10-2025-0006940

(51) Int. Cl. A61L 27/02; A61L 27/20; A61L 27/24; A61L 27/38; A61L 27/50; A61L 27/52; A61L 27/54; A61L 27/56

(52) U.S. Cl. CPC A61L 27/025; A61L 27/20; A61L 27/24; A61L 27/3834; A61L 27/507; A61L 27/52; A61L 27/54; A61L 27/56; A61L 2430/02

(57) ABSTRACT

Described are mesoporous silica for drug delivery, a biocompatible hydrogel composition comprising the same, and the use thereof. The biocompatible hydrogel composition may exhibit the effects of maximizing tissue regeneration and preventing vascular stenosis through sustained drug release by comprising mesoporous silica that has a larger surface area and may exhibit better drug loading properties than conventional porous silica nanoparticles. Also described are an artificial blood vessel made from a bioink comprising the biocompatible hydrogel composition, or a scaffold for artificial tissue regeneration made from the bioink.

### CROSS REFERENCE TO RELATED APPLICATION

The present application claims priority to Korean Patent Application No. 10-2025-0006940, filed Jan. 16, 2025, the entire contents of which are incorporated here for all purposes by this reference.

### BACKGROUND

### 1. Technical Field

The present disclosure relates to a biocompatible hydrogel composition comprising mesoporous silica for drug delivery and the use thereof.

### 2. Related Art

Three-dimensional printing refers to fabricating a complicated skeletal structure by a layer-by-layer process after converting any configuration information obtained from medical data of tissues or organs having complex configurations into G-code. Such three-dimensional printing is also referred to as “three-dimensional bioprinting (3D bioprinting)”. Typically, a biocompatible polymer hydrogel is fabricated into a three-dimensional structure using a piston-type syringe, etc.

“Bioink” is a general term for materials that contain living cells or biomolecules and may be applied to bioprinting technology to fabricate required structures. Therefore, bioink must provide physical properties for 3D processing and a biological environment for cells to perform their intended functions. First of all, bioink should have excellent cell affinity. For artificial tissue and organ regeneration, a biological environment favorable for the proliferation and differentiation of printed cells should be provided from the bioink. When the printing process is long, the cells should be properly supplied with nutrients and oxygen necessary for cell survival in the cartridge. In addition, the cells should be able to be protected from physical stress generated during the printing process.

Materials known as components of bioink include hydrogel. Hydrogel may easily absorb water due to its excellent hydrophilicity, and also its strength and shape may be easily changed, and thus the hydrogel is used as a scaffold for tissue engineering or for drug delivery. In addition, the hydrogel swells by absorbing a large amount of water in an aqueous solution or aqueous environment and due to its hydrophilicity, but has the property of not being dissolved due to its crosslinked structure. Hydrogels having various shapes and properties may be produced depending on the composition and production method thereof, and are generally characterized by having intermediate properties between liquid and solid because they contain a large amount of water.

Biomaterials approved by the FDA include hyaluronic acid, fibrin, and collagen. In general, natural biopolymers such as the above biomaterials have been considered difficult to apply to 3D bioprinting without any treatment.

That is, these biopolymers have limited mechanical properties and lack cell adhesion motifs, and thus their function as scaffolds supporting cell adhesion and proliferation is limited. There is a need to develop a bioink that overcomes the above problems.

### PRIOR ART DOCUMENTS

### Patent Documents

(Patent Document 1) KR 10-2024-0114492 A

### SUMMARY

An object of the present disclosure is to provide a biocompatible hydrogel composition comprising mesoporous silica for drug delivery and the use thereof.

Another object of the present disclosure is to provide a biocompatible hydrogel composition that may exhibit the effects of maximizing tissue regeneration and preventing vascular stenosis through sustained drug release by comprising mesoporous silica that has a larger surface area and exhibit better drug loading properties than conventional porous silica nanoparticles.

Still another object of the present disclosure is to provide an artificial blood vessel made from a bioink comprising the biocompatible hydrogel composition, or a scaffold for artificial tissue regeneration made from the bioink.

To achieve the above-described objects, in one aspect, the present disclosure provides a biocompatible hydrogel composition comprising: spherical mesoporous silica loaded with a drug; sodium alginate; and collagen, wherein the mesoporous silica is capable of releasing the loaded drug in a controlled and sustained manner.

In addition, the inside of the mesoporous silica may include a plurality of mesopores, and the surface of the mesoporous silica may have fine grooves derived from the mesopores.

In addition, the mesopores in the mesoporous silica may have incorporated metal nanoparticles composed of at least one of zinc, zinc oxide, silver, platinum, gold, and mixtures thereof.

In addition, the drug may be selected from the group consisting of small-molecule substances, antibodies, antibiotics, immunosuppressants, anticancer agents, vascular narrowing inhibitors, cell growth promoters, and combinations thereof.

In addition, the mixing ratio between the collagen and the sodium alginate may be 2.5:1 to 5:1.

In addition, the hydrogel composition may further comprise mesenchymal stem cells or tissue progenitor cells.

In another aspect, the present disclosure provides a bioink comprising the biocompatible hydrogel composition.

In still another aspect, the present disclosure provides an artificial blood vessel made from the bioink.

In yet another aspect, the present disclosure provides a scaffold for bone regeneration made from the bioink.

The biocompatible hydrogel composition comprising mesoporous silica according to the present disclosure may exhibit the effects of maximizing tissue regeneration and preventing vascular stenosis through sustained drug release by comprising mesoporous silica that has a larger surface area and may exhibit better drug loading properties than conventional porous silica nanoparticles.

The present disclosure also provides an artificial blood vessel made from a bioink comprising the biocompatible hydrogel composition, or a scaffold for artificial tissue regeneration made from the bioink.

### BRIEF DESCRIPTION OF THE DRAWINGS

FIGS. 1A-1F show TEM images (1A), size distributions (1B), zeta potentials (1C) of nanoparticles (NP) and drug-loaded nanoparticles (NPC and NPS), TEM images and size distributions after long-term storage (1D and 1E) of NP, and drug release patterns of NPC and NPS (1F).

FIGS. 2A-2K show an in vitro experiment for evaluating the cytotoxicity of drug-loaded nanoparticles (NPC, NPS, and NPSC) (2A), the results of evaluating cytotoxicity depending on the treatment concentration of mesoporous silica (2B), the results of evaluating cytotoxicity following treatment with each type of nanoparticles (2C to 2E), the results of evaluating tube formation (2F to 2I), and the results of evaluating antioxidant effect (2J and 2K).

FIGS. 3A-3J show a schematic view of artificial blood vessels (3A), the configuration of a coaxial nozzle of a 3D bioprinter (3B), various types of artificial blood vessels prepared by 3D bioprinting (3C), vascular endothelial progenitor cells included in artificial blood vessels (3D and 3F), and the results of evaluating the durability of the prepared artificial blood vessels (3G and 3H).

FIGS. 4A-4I show a summary of an experiment using a mouse hindlimb ischemia model (4A), blood flow analysis and surgical site images of each experimental group (4B to 4E), and the results of analyzing the expression of vascular-related markers (α-SMA and CD31) (4F to 4I).

FIG. 5 shows a method for fabricating a scaffold for bone formation.

FIGS. 6A-6D show a summary of an experiment using a mouse calvarial defect model (6A) and the results of analyzing bone regeneration at the implantation site by MCT (6B and 6C).

FIGS. 7A-7G show the results of histological analysis of a new bone formation area in a mouse calvarial defect model (7A to 7C) and the results of analysis of vascular markers (7D to 7G).

FIGS. 8A-8B show images of angiogenesis in a mouse calvarial defect model and the results of volume analysis.

FIGS. 9A-9E show a summary of an experiment using a mouse hindlimb ischemia model (9A), and the results of blood flow analysis for each experimental group and images of the surgical site (9B to 9E).

FIGS. 10A-10D show the results of analyzing the distribution of M1/M2 macrophages in the results of an experiment using a mouse hindlimb ischemia model (10A to 10D).

### DETAILED DESCRIPTION

Hereinafter, examples of the present disclosure will be described in detail so that the present disclosure can be easily carried out by those skilled in the art. However, the present disclosure may be embodied in a variety of different forms and is not limited to the example described herein.

It should be understood that although certain aspects herein are described with the term “comprising . . . ”, other similar aspects described in terms of “consisting of . . . ” and/or “consisting essentially of . . . ” are also provided.

### Biocompatible Hydrogel Composition

Materials are of utmost importance in the fabrication of scaffolds for tissue regeneration. In particular, in the field of artificial blood vessels, research on which is most active, synthetic polymer compounds selected from the group consisting of polyurethane (PU), silicone, polyethylene terephthalate (PET), polytetrafluoroethylene (PTFE), expanded polytetrafluoroethylene (ePTFE), polydioxanone (PDO), poly(L-lactide) (PLLA), polyglycolic acid (PGA), and poly(ε-caprolactone) (PCL) have been used alone or in combination.

However, since all the materials mentioned above are not of a biological origin, they are likely to cause inflammation, and since cells cannot adhere thereto, artificial blood vessels made from the materials can only be substitutes for problematic blood vessels and cannot serve as scaffolds for regenerating blood vessels.

Therefore, there have been attempts to replace the synthetic polymer materials mentioned above with natural polymers such as gelatin, collagen, elastin, fibrin, chitin, chitosan, hyaluronic acid, heparin, or laminin, but it is known to be difficult to obtain the desired durability.

However, despite these various reports, the inventors of the present disclosure have invented a biocompatible hydrogel having both desired durability and cell adhesion ability/cell growth ability by mixing collagen and sodium alginate at a ratio of 2.5:1 to 5:1.

The viscosity of the sodium alginate is 2,000 cP or higher at room temperature. If the viscosity is lower than 2,000 cP, the desired physical strength cannot be obtained.

The mixing ratio between the collagen and the sodium alginate may be selected from 2.5:1, 2.7:1, 3:1, 3.3:1, 3.5:1, 3.7:1, 4:1, 4.3:1, 4.5:1, 4.7:1, and 5:1. If the mixing ratio is less than 2.5 (collagen):1 (sodium alginate), cell adhesion may not be efficient, and if the mixing ratio is more than 5 (collagen):1 (sodium alginate), the physical strength of the mixture will be lower than the desired strength, and thus the mixture may not be used as a scaffold.

The hydrogel composition of the present disclosure may comprise a spherical mesoporous silica loaded with a drug. When the hydrogel composition comprises a drug, there is an advantage in that the drug may be delivered directly to the implantation site. However, there are disadvantages in that, since the release rate of the drug may not be controlled at a constant level, cytotoxicity may be induced by a high concentration of the drug in the initial stage of implantation, and after the initial stage, the drug is completely released and no longer exhibits an effect. Therefore, the hydrogel composition of the present disclosure is characterized by comprising a mesoporous silica that may be loaded with a high dose of a drug and may release the drug in a sustained manner by controlling the release rate at a constant level.

The mesoporous silica nanoparticles may have an average diameter of 10 nm to 500 nm, a specific surface area of 500 m²/g to 2,000 m²/g, and an average mesopore diameter of 2 nm to 25 nm.

As described above, the mesoporous silica of the present disclosure shows large differences in the specific surface area, average pore diameter, and pore volume from conventional mesoporous silica. Due to these differences in the specific surface area, average pore diameter, and pore volume, even if the same amount of the mesoporous silica as conventional mesoporous silica is used, the mesoporous silica of the present disclosure shows a large difference in the area where a reaction by contact may occur. Accordingly, since the mesoporous silica of the present disclosure has a large specific surface area, average pore diameter, and pore volume as described above, the amount of drug that may be loaded into the mesopores may increase, and drug loading is possible without being significantly affected by the particle size of the drug.

In addition, the mesopores in the mesoporous silica may have incorporated metal nanoparticles composed of at least one of zinc, zinc oxide, silver, platinum, gold, and mixtures thereof.

Preferably, in the mesopores in the mesoporous silica, zinc ions may be bonded to silica to form zincosilicate.

The zincosilicate comprises a bonded structure represented by Formula 1 below:

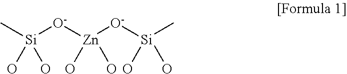

As shown in Formula 1 representing the structure of zincosilicate, the oxygen atom bonded to Si is negatively charged, a zinc ion (Zn²⁺) is bonded to the oxygen atom, and the bonded zinc ion may be oxidized to form zinc oxide. As zinc oxide is bonded to the inside of the mesoporous silica, the effect of the bounded zinc oxide as well as the effect of the mesoporous silica may be exhibited together.

That is, as zinc oxide is incorporated in the mesopores as described above, chemical bonding between the incorporated zinc oxide and a drug may occur, thereby further enhancing the drug loading effect.

In addition to the mesoporous silica having zinc oxide bonded to the inside of the mesopores as described above, mesoporous silica having zinc and silver nanoparticles incorporated in the mesopores, or mesoporous silica having zinc and gold nanoparticles incorporated in the mesopores, may be used.

The mesoporous silica included in the biocompatible hydrogel composition may be either mesoporous silica having no metal nanoparticles incorporated therein (SMB-3), mesoporous silica having a structure in which zinc ions are bonded to form zincosilicate (SMB-7), mesoporous silica having zinc and silver nanoparticles incorporated therein (SMB-24), or mesoporous silica having zinc and gold nanoparticles incorporated therein (SMB-73).

More specifically, the SMB-3 has a pure silica-based mesoporous structure, may be loaded with a drug, and may have the maximized property of releasing the drug in a sustained manner. In particular, the SMB-3 has high biodegradability and biocompatibility, making it suitable for long-term drug delivery. The SMB-7 has a mesoporous structure including a zincosilicate structure, has incorporated therein zinc quantum dots in an amount of 5 wt % based on the total weight of the mesoporous silica, and has the property of promoting anti-inflammation and cell proliferation. The SMB-7 may provide vascular regeneration and anti-inflammatory effects. The SMB-24 has a structure containing zinc quantum dots and silver nanoparticles in amounts of 5 wt % and 3 wt %, respectively, based on the total weight of the mesoporous silica, and has excellent antibacterial activity and drug delivery properties. The SMB-24 may be useful mainly for infection prevention and treatment. The SMB-73 has a structure containing zinc quantum dots and gold nanoparticles in amounts of 5 wt % and 1 wt %, respectively, based on the total weight of the mesoporous silica, is suitable for delivery of a high value-added drug such as an anticancer drug, and has a structure capable of maximizing the anticancer effect.

The mesoporous silica having metal nanoparticles incorporated therein as described above may not only facilitate chemical bonding of a drug thereto by the metal nanoparticles, but also exhibit the effects of zinc, silver, and/or gold. That is, antibacterial and antioxidant effects, etc., may be exhibited by the metal nanoparticles.

The mesoporous silica is spherical in shape, is not toxic by itself, and maintains its structure for one week or more, and the drug loaded therein is slowly released over a period of about a week. Therefore, although the concentration of the drug loaded in the mesoporous silica is higher than the general drug treatment concentration, it has the advantage of not exhibiting direct toxicity caused by the drug. In addition, since the doubling time of cells is known to be generally 1 to 3 days, drug release over one week period may directly affect the vascular endothelial progenitor cells contained in artificial blood vessels. Therefore, the vascular endothelial progenitor cells contained in the artificial blood vessels are able to divide and differentiate through sustained drug release, thereby enabling blood vessel regeneration.

The type and form of drug that may be loaded in the mesoporous silica of the present disclosure are not limited. The drug may be selected from the group consisting of small-molecule substances, antibodies, and combinations thereof.

In addition, the drug may be selected from the group consisting of antibiotics, immunosuppressants, vascular stenosis inhibitors, cell growth promoters, and combinations thereof, depending on the intended use.

The antibiotic may be selected from the group consisting of chlorhexidine gluconate, chlorohexidin acetate, chlorhexidine hydrochloride, silver sulfadiazine, povidone iodine, chlortetracycline, neomycin, penicillin, gentamicin, polymyxin B, ciprofloxacin, fusidic acid, ofloxacin, tobramycin, and combinations thereof.

The immunosuppressant may be selected from the group consisting of cyclosporine, azathioprine, mycophenolic acid, mycophenolate mofetil, corticosteroid, prednisone, methotrexate, sulfasalazine, leflunomide, mizoribine, cyclophosphamide, rapamycin, tacrolimus, and combinations thereof.

The vascular stenosis inhibitor may be selected from the group consisting of paclitaxel, docetaxel, cabazitaxel, rapamycin, tacrolimus, pimecrolimus, temsirolimus, everolimus, biolimus, zotarolimus, and combinations thereof.

The cell growth promoter may be selected from the group consisting of curcumin, avastatin, atorvastatin, dexamethasone, dexamethasone phosphate, dexamethasone acetate, StemRegenin (SR1), and combinations thereof.

In one example of the present disclosure, at least one selected from the group consisting of curcumin, atorvastatin, SR1, and combinations thereof was used as a cell growth promoter.

In one example of the present disclosure, at least one selected from the group consisting of paclitaxel, rapamycin, and a combination thereof was used as a vascular stenosis inhibitor.

The hydrogel composition of the present disclosure may further comprise mesenchymal stem cells or tissue progenitor cells for tissue regeneration. The mesenchymal stem cells may be derived from bone marrow, umbilical cord, adipose tissue, or peripheral blood, without being limited thereto. The tissue progenitor cells may be vascular endothelial progenitor cells, adipose progenitor cells, liver progenitor cells, neural progenitor cells, muscle progenitor cells, skin progenitor cells, or joint progenitor cells, without being limited thereto.

In one embodiment of the present disclosure, the hydrogel composition comprises vascular endothelial progenitor cells isolated and proliferated from an umbilical cord. However, the hydrogel composition of the present disclosure does not necessarily comprise the vascular endothelial progenitor cells.

### Scaffold for Preparation of Artificial Blood Vessels Made of Biocompatible Hydrogel Composition

The hydrogel composition of the present disclosure may be used as a bioink for scaffold fabrication. In the present disclosure, for the preparation of artificial blood vessels, cylindrical scaffolds were fabricated using a 3D bioprinter equipped with a coaxial nozzle using a core-shell bioink as a material. Regarding the core bioink, 40% wt/v Pluronic F-12 containing 100 mM calcium chloride was used as a sacrificial material. Cylindrical scaffolds having various outer and inner diameters were fabricated by adjusting the diameters of the inner and outer needles of the coaxial nozzle as needed. The outer diameter of the cylindrical scaffold may be 3 mm to 250 μm μm, and the inner diameter may be 2.5 mm to 100 μm, so that the cylindrical scaffold may be fabricated according to the diameter and inner diameter of the artificial blood vessel to be transplanted.

Therefore, the cylindrical scaffold of the present disclosure has a single-layer structure. The single-layer structure not only has a simple fabrication process compared to a multilayer structure, but also maintains uniform mechanical properties, which may increase the stability of the scaffold. In addition, the single-layer structure provides an environment in which cells can easily penetrate and grow, which is advantageous for tissue regeneration. In addition, the single-layer structure allows for smoother material exchange than the multi-layer structure, which enables efficient supply of nutrients and oxygen, and it has the effect of reducing costs by using less material in the fabrication process. Finally, the single-layer structure is able to more easily control biological reactions, which is advantageous in designing therapeutic scaffolds according to specific tissues or organs.

Artificial Blood Vessel Made from Biocompatible Hydrogel Composition

The cylindrical scaffold fabricated by 3D printing in the present disclosure may be trimmed and used directly as an artificial blood vessel.

The artificial blood vessel of the present disclosure comprises vascular endothelial progenitor cells and curcumin and statin loaded in the mesoporous silica. The two drugs may be loaded simultaneously in a single nanoparticle or loaded separately.

As the artificial blood vessel of the present disclosure comprises vascular endothelial progenitor cells, the durability thereof is at least 3 times higher than that of an artificial blood vessel made from a hydrogel that does not comprise vascular endothelial progenitor cells. The concentration of vascular endothelial progenitor cells included in the artificial blood vessel of the present disclosure is 0.1×10⁶ to 5×10⁶ cells/ml. If the concentration of the precursor cells is less than 0.1×10⁶ cells/ml, vascular regeneration is not achieved properly, and if the concentration is more than 5×10⁶ cells/ml, there is a disadvantage in that the cells affect the physical properties of the prepared artificial blood vessel.

It was confirmed that the artificial blood vessel containing curcumin and statin loaded in the mesoporous silica and vascular endothelial progenitor cells according to the present disclosure not only successfully restored blood flow in a mouse hindlimb ischemia model, and that the vascular endothelial progenitor cells present in the artificial blood vessel proliferated so that the blood vessel was intrinsically regenerated.

In addition, it was confirmed that, when an artificial blood vessel containing nanoparticles loaded with paclitaxel or rapamycin, which is known to inhibit vascular stenosis, was transplanted into a mouse hindlimb ischemia model, it not only restored blood flow as in the artificial blood vessel transplantation results mentioned above, but also increased the distribution of M2 macrophages, which are anti-inflammatory macrophages, at the artificial blood vessel transplantation site.

Stimulation of angiogenesis has been highlighted as a key strategy in regenerative medicine to stimulate the repair of damaged tissues such as bone, cartilage, muscle, and nerves by supplying sufficient nutrients and oxygen.

Therefore, it can be seen that the artificial blood vessel of the present disclosure not only restores blood flow immediately after transplantation, but also has a long-term vascular regeneration effect.

In the present disclosure, the scaffold fabricated as described above was arranged in a multilayer grid pattern to fabricate a patch-type scaffold. This multilayer grid-type patch has an increased surface area, which enables efficient release of a drug loaded in the nanoparticles.

In the present disclosure, a scaffold for bone grafting was fabricated by this fabrication method. The scaffold comprises SR1 loaded in the mesoporous silica nanoparticles. The SR1 is an aryl hydrocarbon receptor (AHR) antagonist known to induce CD34+ hematopoietic stem cell proliferation.

In the present disclosure, as a result of transplanting the bone scaffold of the present disclosure into a mouse calvarial defect model, it was confirmed that, in the group transplanted with the scaffold containing the nanoparticles loaded with SR1, not only was the bone tissue at the defect site almost regenerated, but also the vascular network around the scaffold was regenerated.

Therefore, it can be seen that the bone scaffold of the present disclosure may ultimately be used in regenerative medicine to induce not only bone tissue regeneration but also vascular regeneration.

Hereinafter, the present disclosure will be described in more detail by way of experimental examples and examples. These examples are only intended to explain the present disclosure in more detail, and it will be apparent to those skilled in the art that the scope of the present disclosure according to the subject matter of the present disclosure is not limited by these examples.

### Experimental Example 1. Method for Producing Drug-Loaded Mesoporous Silica Nanoparticles (NP)

First, 1 mmol of dodecylamine (DDA) was added to 20 mL of a 10% aqueous solution of ethanol, followed by stirring for 1 hour at 60±1° C. until the solution became transparent. Next, the solution was kept with stirring at room temperature for 1 hour. Then, 5 mL of an aqueous solution containing zinc nitrate ions was added thereto, followed by for 1 hour. Thereafter, 4 mmol of tetraethylorthosilicate (TEOS), a silica precursor, was added to the solution, followed by stirring at room temperature (20 to 25° C.) for 1 hour to form a mesoporous silica structure. Thereafter, 0.2 mmol of NaBH₄, a reducing agent, was added to the stirred solution to obtain spherical mesoporous silica, which was then vacuum-filtered at a pressure of 30 mmHg. Next, the filtered spherical mesoporous silica was washed three times with 200 ml of distilled water and three times with ethanol at 60° C. After completion of the washing process, the mesoporous silica was dried at 70° C. for 2 hours and calcined at 550° C. for 6 hours, thereby producing SMB-7.

Meanwhile, tetraethylorthosilicate (TEOS), a silica precursor, was added to a IN aqueous solution of hydrochloric acid, stirred for 2 hours, filtered, washed three times with 200 ml of distilled water, and then dried at 70° C. for 5 hours, thereby producing SMB-3.

To produce drug-loaded mesoporous silica, 80 mg of SMB-7 or SMB-3 was dissolved in 80 ml of ethanol and then dispersed by ultrasonication for 3 minutes. Depending on the properties of a drug, the drug was dissolved in ethanol or DMSO to prepare a drug solution, and then the mesoporous silica solution and the drug solution were mixed and kept at a pressure of 100 bar for 1 hour in a vacuum. Next, the pressure was lowered to 60 bar, and the solution was left at that pressure for 30 minutes and then dried in a vacuum at room temperature for 6 hours, thereby producing drug-loaded mesoporous silica.

The drug solutions used in the examples below were prepared by dissolving 20 mg of each of atorvastatin, curcumin, and rapamycin in 20 mL of ethanol, and the StemRegenin-1 (SR1) solution was prepared by dissolving 25 mg of SR1 in 1 mL of DMSO.

### Experimental Example 2. Method for Producing Vascular Endothelial Progenitor Cells

Human endothelial progenitor cells (hEPCs) were isolated from human umbilical cord blood (hUCB) according to a previously reported method. Specifically, hUCB was collected from healthy volunteers after obtaining written consent according to a protocol approved by the Institutional Review Board of Pusan National University Yangsan Hospital (Approval No.: PNUYH-05-2017-053). Mononuclear cells isolated from hUCB were cultured in endothelial growth medium-2 (EGM-2) containing 5% fetal bovine serum (FBS), human vascular endothelial growth factor (hVEGF), human basic fibroblast growth factor (hbFGF), human epidermal growth factor (hEGF), human insulin-like growth factor 1 (hIGLGF), ascorbic acid, and GA-1000 (Lonza, Walkersville, MD, USA). After 4 days of culture, non-adherent cells were removed, and the attached cells were cultured for a long period of time to form spindle-shaped colonies, and the medium was replaced every 14 to 21 days. Several surface and key functional markers were characterized by flow cytometry. EPCs are positive for endothelial lineage markers (CD31 and VEGFR2) and hematopoietic stem cell markers (CD34, CXCR4, c-Kit), and negative for hematopoietic lineage markers such as CD11b, CD14, and CD45.

### Experimental Example 3. Method for Preparing Hydrogel Bioink (Shell Bioink)

To prepare a hydrogel bioink, sodium alginate (viscosity >2,000 cP, 25° C., Sigma-Aldrich) was dissolved in DPBS, followed by stirring at 37° C. for 6 hours. Atelocollagen solution (pH 4.0, Baobab Healthcare, Gyeonggi-do, Korea) was neutralized by mixing with reconstituted buffer (132 mM Na₂HPO₄) at a 1:1 volume ratio.

### Experimental Example 4. Method for Fabricating Scaffold for Preparing Artificial Blood Vessel

Cylindrical scaffolds for tissue regeneration were fabricated using a coaxial nozzle (inner needle 28 G; outer needle 20 G; Ramé-hart, Succasunna, NJ, USA) provided in a 3D bioprinter (Root 1; Baobab Healthcare, Gyeonggi-do, Republic of Korea). Here, cylindrical scaffolds with various outer/inner diameters were fabricated by controlling the diameters of the inner and outer needles of the coaxial nozzle. Shell bioinks were prepared by mixing 3% wt/v neutralized atelocollagen and 3% wt/v sodium alginate at a certain ratio (2.5:1 to 5:1). Thereafter, the drug-loaded mesoporous silica containing vascular endothelial progenitor cells (1×10⁶ cells/ml) or the drug-loaded mesoporous silica without vascular endothelial progenitor cells was mixed with the shell bioinks. Regarding the core material, 40% wt/v Pluronic F-12 (Sigma-Aldrich) containing 100 mM calcium chloride was used as a sacrificial material.

The core and shell bioinks were bioprinted at 80 kPa (core) and 60 kPa (shell) using a 200 mM CaCl₂) solution to enable in situ crosslinking of the alginate solution. The sacrificial material (core bioink) was removed using cold DPBS. The fabricated scaffolds have a hollow cylindrical shape and may be used directly for vascular grafting. Alternatively, the scaffold may be overlapped in a multilayer grid pattern and used as a scaffold for bone grafting.

### Experimental Example 5. Mouse Hindlimb Ischemia Model

BALB/CA-nu/nu athymic immunodeficient mice were used as a hindlimb ischemic animal model to be transplanted with 3D-printed artificial blood vessels. Animal experiments were performed using a protocol approved by the Pusan National University Institutional Animal Use and Care Committee (Approval No.: PNU-2022-0212). All mice were anesthetized by intraperitoneal injection of 2,2,2 tribromoethanol (400 mg/kg; Avertin, Sigma-Aldrich) and underwent surgery for femoral artery resection and laser Doppler perfusion imaging. One femoral artery per animal was excised from its proximal origin as a branch of the external iliac artery to its distal bifurcation into the saphenous and popliteal arteries. Immediately after artery ligation, an artificial blood vessel was transplanted into the hindlimb where ischemia occurred.

After ischemia was induced in the mouse hindlimb ischemia model, angiogenesis was observed at four time points (0, 3, 7, and 14 days) using laser Doppler perfusion imaging (LDPI; Moor Instruments Ltd, Devon, UK). The blood perfusion level was measured by comparing the perfusion status of the normal right limb with that of the ischemic left limb at each time point. The degree of necrosis was assessed by observing the mice on days 0 and 14 after ischemia induction, and the degree of necrosis of each animal was used for quantitative analysis.

For tissue immunostaining, thigh tissues were harvested, fixed in formalin, embedded in paraffin, and sectioned to 5 μm thickness. Blood vessels were stained with an antibody against alpha smooth muscle actin (ab5694; 1:100 Abcam plc., Cambridge, UK), and nuclei were stained with DAPI using ProLong Diamond Antifade Mountant with DAPI (Invitrogen). Stained sections were visualized using a Lionheart FX automated microscope (BioTek, Winooski, USA).

### Experimental Example 6. Rat Calvarial Defect Model

Twenty-eight female Sprague-Dawley rats (age of 3 months, body weight of 240±20 g) were purchased from Koatech (Pyeongtaek, Republic of Korea). Animal experiments were performed according to the methods approved by the Institutional Animal Care and Use Committee (IACUC) of Daegu-Gyeongbuk Medical Innovation Foundation (Approval No.: KMEDI-22080801-01).

The 28 rats were divided into three groups according to body weight. The animals were anesthetized by intraperitoneal injection of Zoletil (50 mg/mL) and rompun (23.32 mg/mL), and 5-mm diameter defects were created in the cranial bone, and then scaffolds or PBS were implanted. After surgery, meloxicam (0.2 mg/kg) as an analgesic was administered subcutaneously. The animals were sacrificed for evaluation 2 and 4 weeks after surgery.

Half of the animals in each group were anesthetized and subjected to micro-computerized tomography (MCT) scanning to evaluate bone regeneration and to visualize the whole cranial bone. The Quantum FX (PerkinElmer, Waltham, Massachusetts, USA) was used to perform MCT scanning 2 and 4 weeks after implantation.

Four weeks after implantation, half of the animals in each group were perfused with Microfil compound (MICROFIL® MV-122; Flow Tech, Cheonan, Republic of Korea) to evaluate blood vessel formation using MCT scanning. The animals were anesthetized, and then 0.2 mL of heparin (5000 IU/mL) was injected intravenously into their tail vein. The animals were then fixed on a polystyrene plate, and the heart was exposed by incising along their bodies' midline. An incision (<1 cm) was made in the left ventricle, and a feeding tube was inserted into their aorta. Thereafter, the blood vessels were flushed by cardiac perfusion using 200 mL of heparin (1 IU/mL)-containing normal saline. The right atrium was incised to allow the perfused normal saline to flow out. Next, 20 mL of Microfil compound was used to perfuse the blood vessels in the same manner with a pressure of 120 mmHg and a perfusion rate of 2 mL/min. Then, the animals were stored overnight at 4° C. to complete the primary setting of Microfil. Next, the animals were fixed in 10% buffered formaldehyde solution for a week and were soaked in 14% EDTA for decalcification for 3 weeks before MCT scanning. Quantum FX (PerkinElmer, Waltham, Massachusetts, USA) was used to perform MCT scanning of the Microfil compound-perfused and decalcified rat calvarial samples.

All animals were sacrificed immediately after MCT scanning, and cranial bone samples were extracted from the sacrificed animals. Harvested samples were fixed in 10% buffered formaldehyde solution for a week and were also decalcified with 14% EDTA for 3 weeks. Next, the samples were cut horizontally, dehydrated, embedded in paraffin, and sectioned to 4 μm thickness. The sections were stained with hematoxylin-eosin (H&E) and Masson's trichrome (MT). Quantitative evaluation of the percentage area of new bone to the total defective area was conducted based on the results of the H&E- and MT-stained slides at 2 and 4 weeks after implantation.

Immunohistochemistry and immunofluorescence were conducted on the obtained cranial slides to analyze the expression of CD31 and a-smooth muscle actin (α-SMA)

### Example 1-1. Characterization of Mesoporous Silica (SMB-3) Loaded with Atorvastatin and Curcumin Drugs

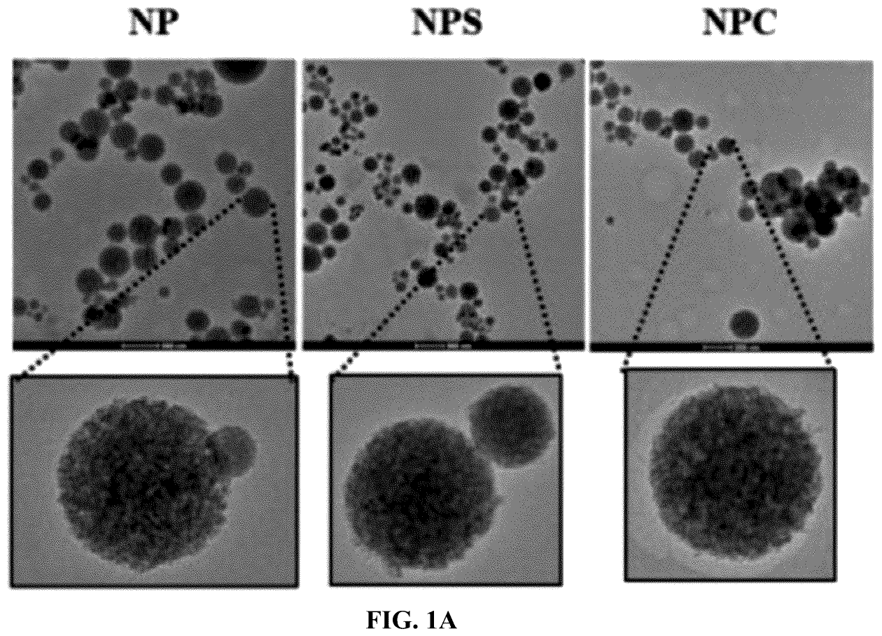

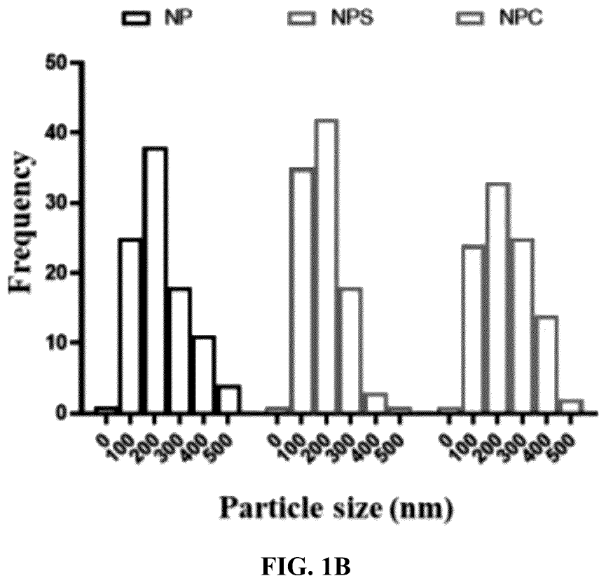

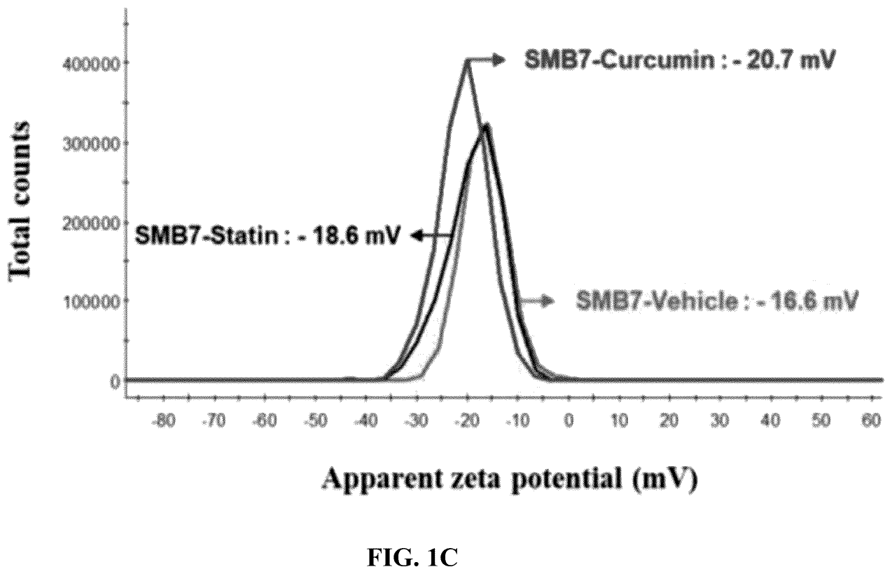

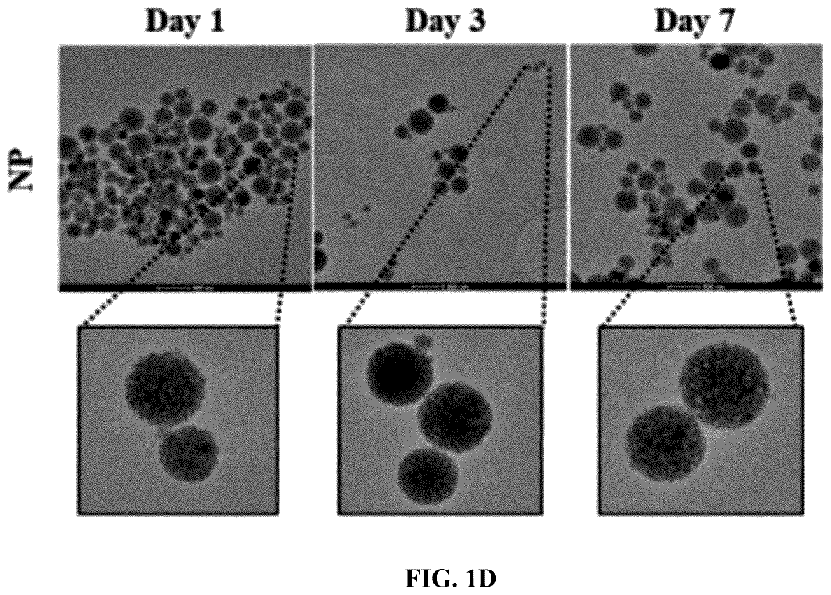

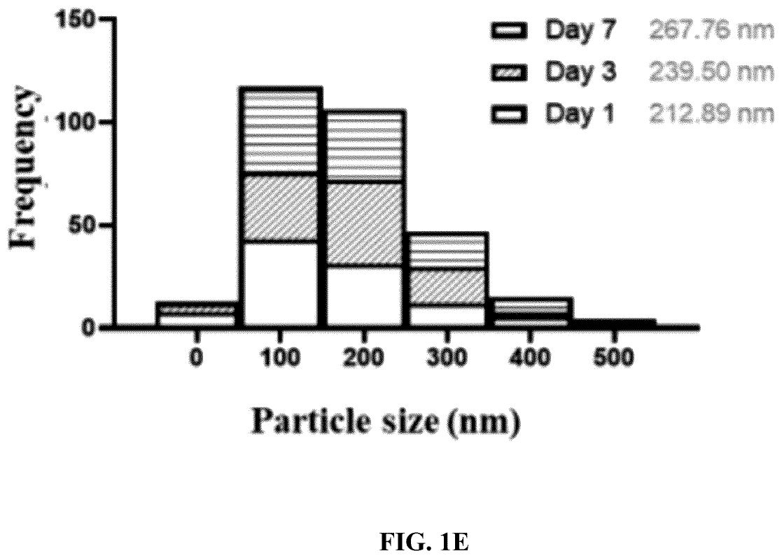

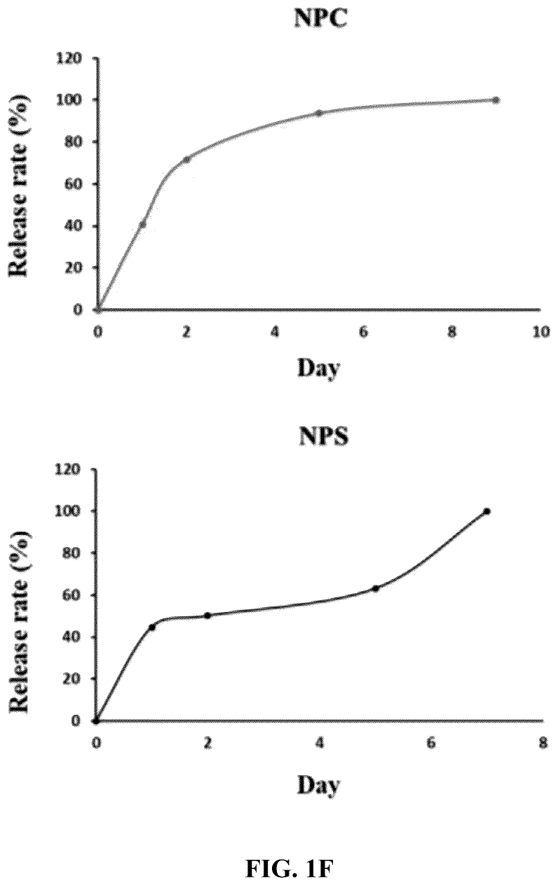

As a result of performing transmission electron microscopy (TEM) image analysis to characterize the mesoporous silica (NP, SMB-3), atorvastatin-loaded mesoporous silica (NPS), and curcumin-loaded mesoporous silica (NPC) produced in Experimental Example 1, it was confirmed that each mesoporous silica maintained a constant spherical diameter, and the size distribution could be confirmed (FIGS. 1A and 1B). In addition, the stability of the nanoparticles was confirmed through zeta potential analysis (FIG. 1C). As a result of analyzing the size after storage at 37° C., it was confirmed that each mesoporous silica maintained a stable size over time (FIGS. 1D and 1E), and as a result of analyzing the cumulative release of the loaded drug, it was confirmed that the drug was released in a sustained manner until about day 7 (FIG. 1F).

### Example 1-2. Evaluation of Biocompatibility of NPS and NPC

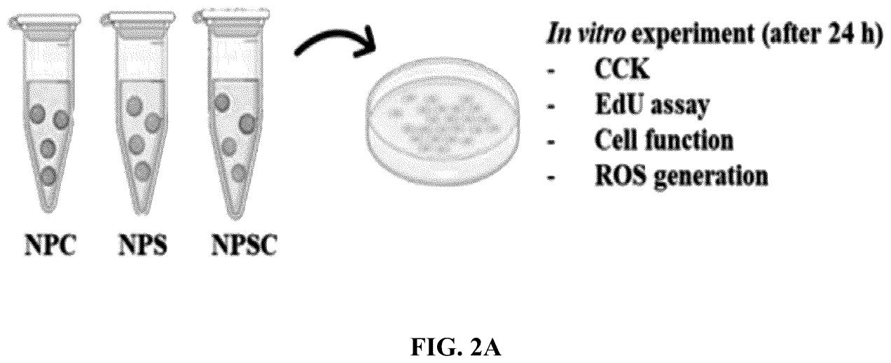

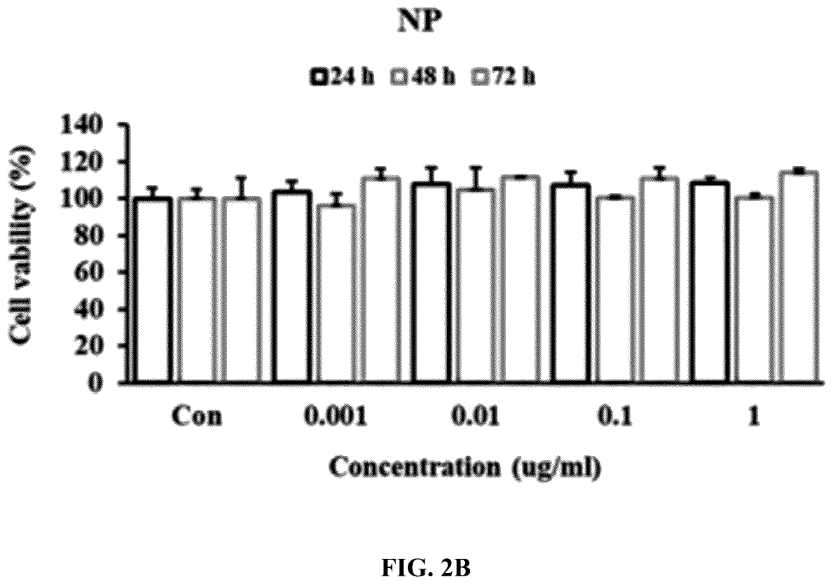

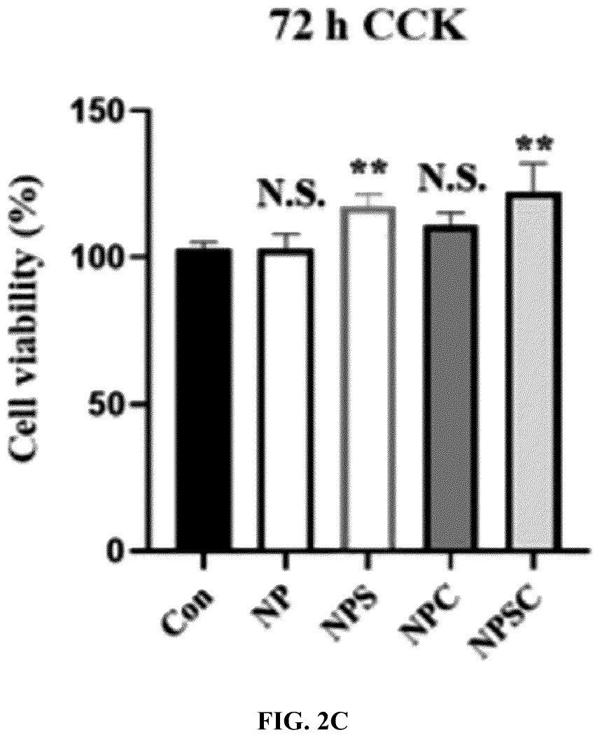

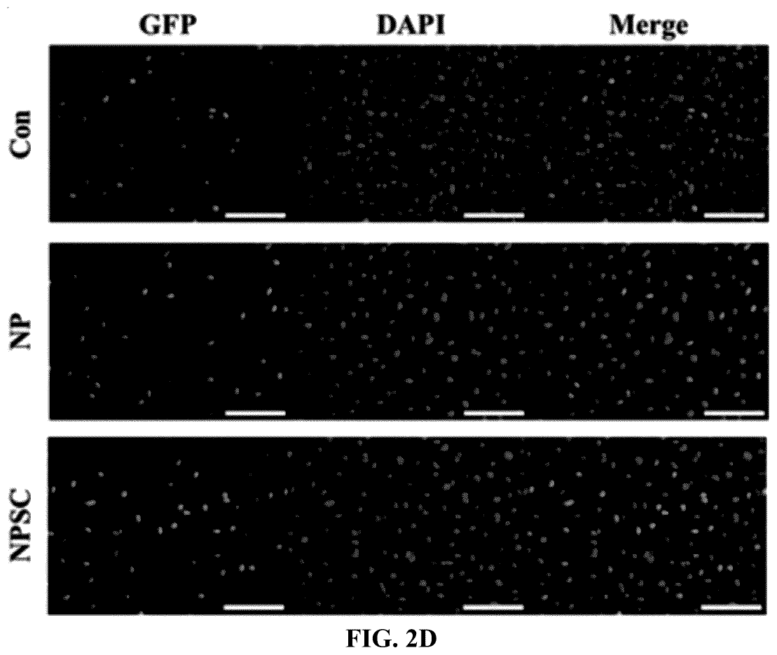

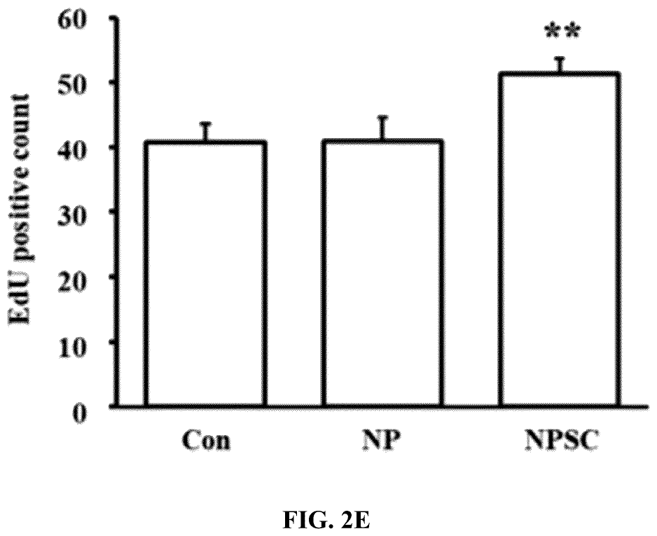

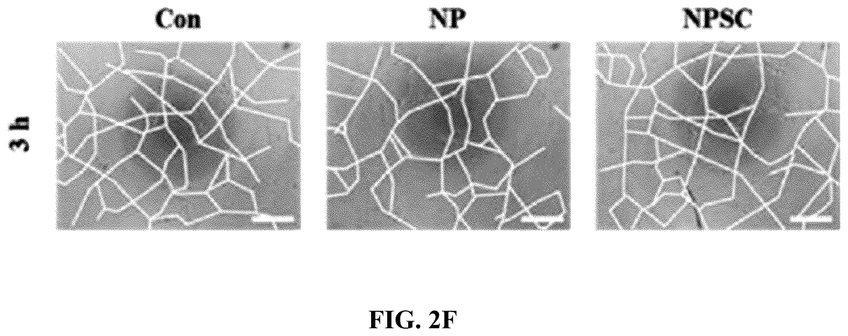

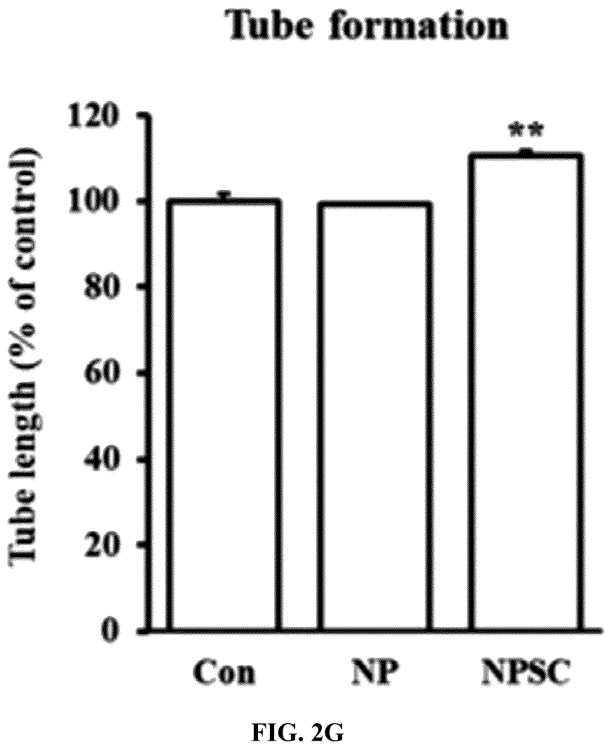

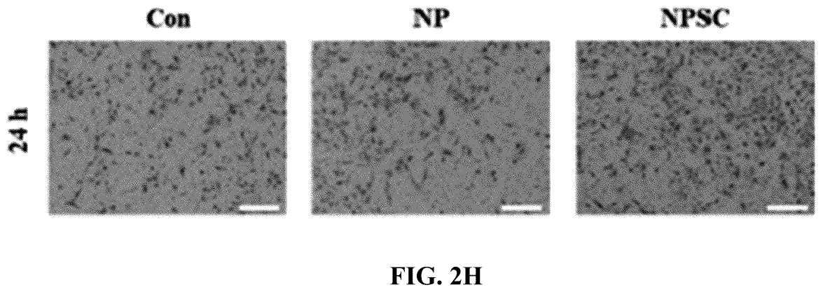

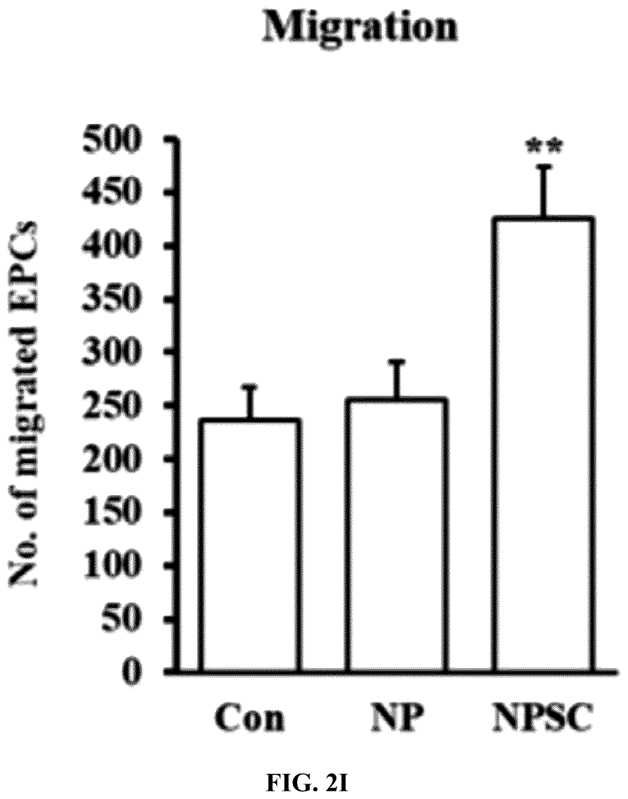

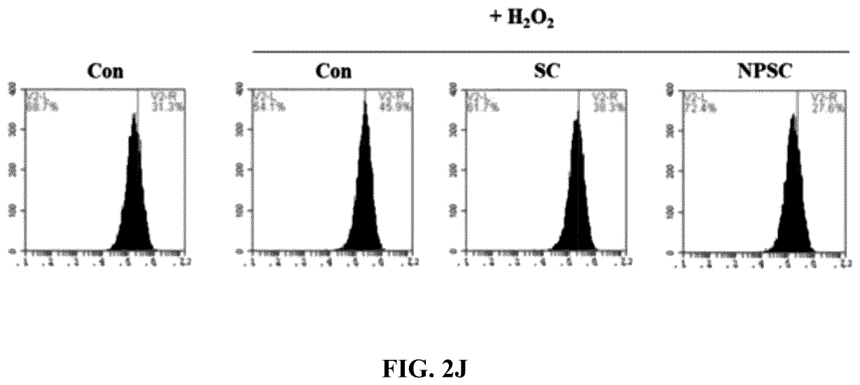

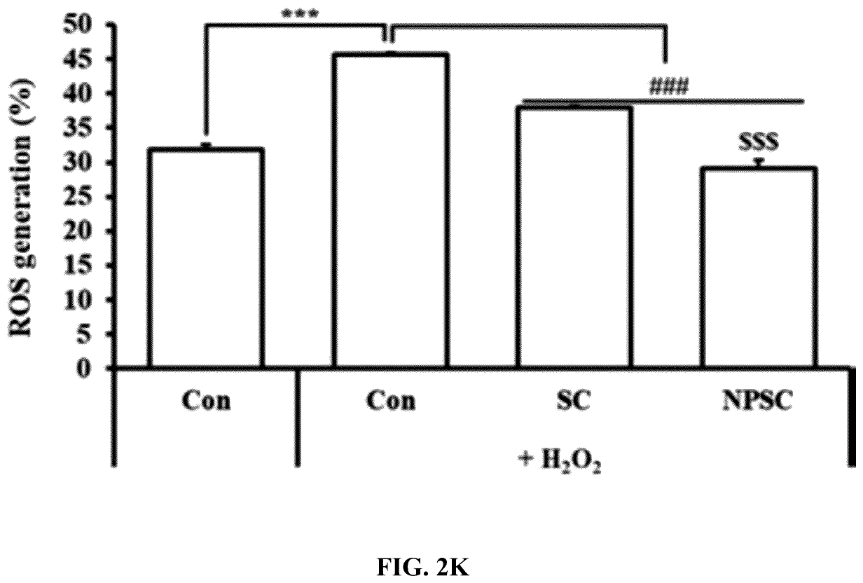

To analyze the effect of the produced mesoporous silica (SMB-7) on vascular endothelial progenitor cells, isolated vascular endothelial progenitor cells were treated with each of NP, NPC, NPS, and NPSC (NPS+NPC) (FIG. 2A). As a result, it was confirmed that no toxicity of mesoporous silica (NP) itself was detected even at a high concentration of 1 mg/ml (FIG. 2B), and that treatment with the combination of NPS and NPC (NPSC) showed the highest cell viability (FIGS. 2C to 2E). It was confirmed that NPSC treatment increased the tube-forming ability and cell migration ability of the vascular endothelial progenitor cells (FIGS. 2F to 2I), and also showed resistance to oxidative stress (H₂O₂) (FIGS. 2J and 2K).

Therefore, it can be seen that the combination of NPS and NPC (NPSC) has a remarkable effect on improving the activity, viability, and cell proliferation of vascular endothelial progenitor cells compared to each of NPC and NPS.

### Example 1-3. Preparation of 3D-Printed Artificial Blood Vessels and Characterization Thereof Through Evaluation of Cell Viability

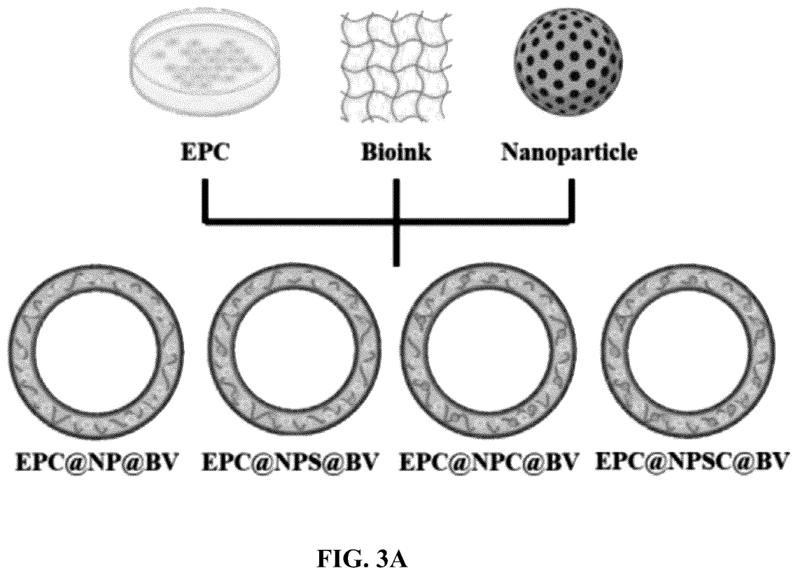

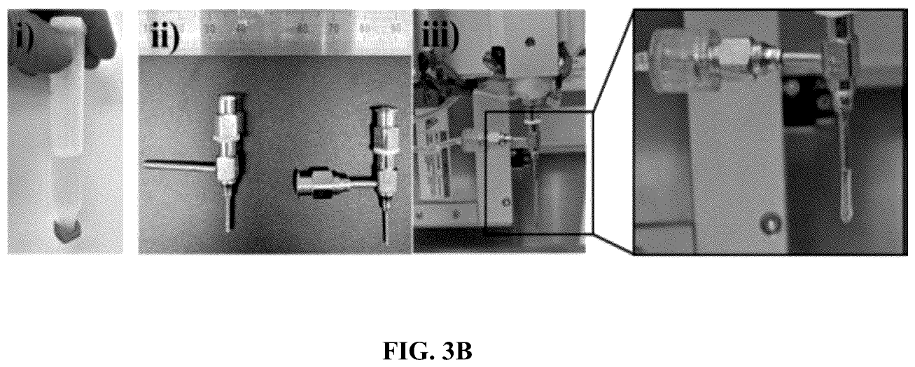

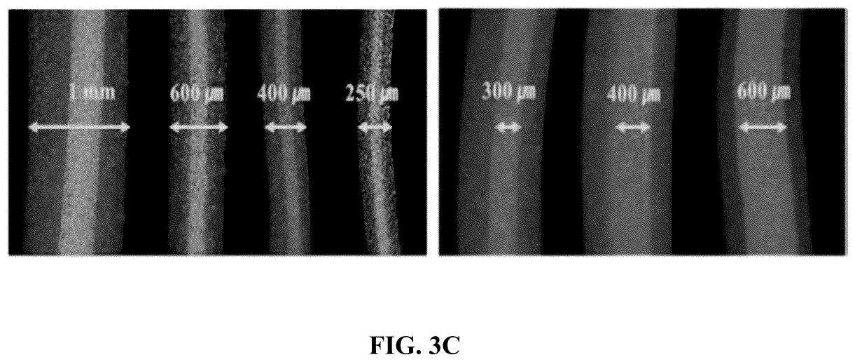

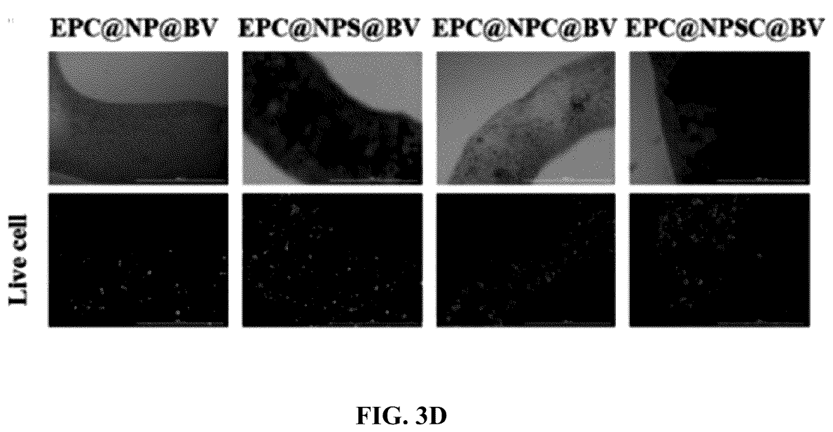

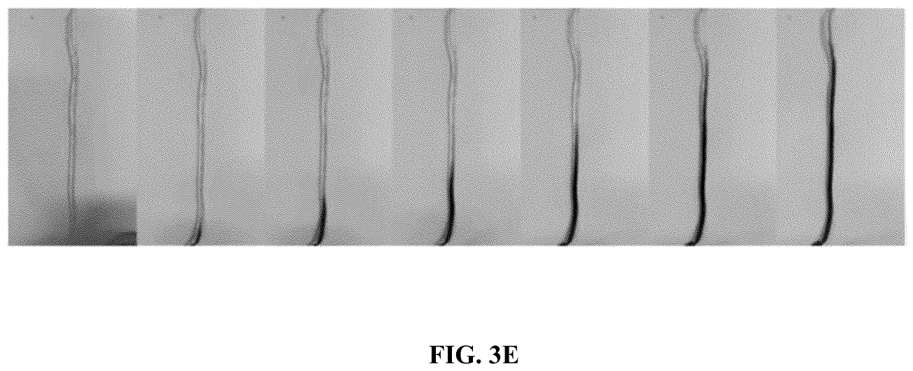

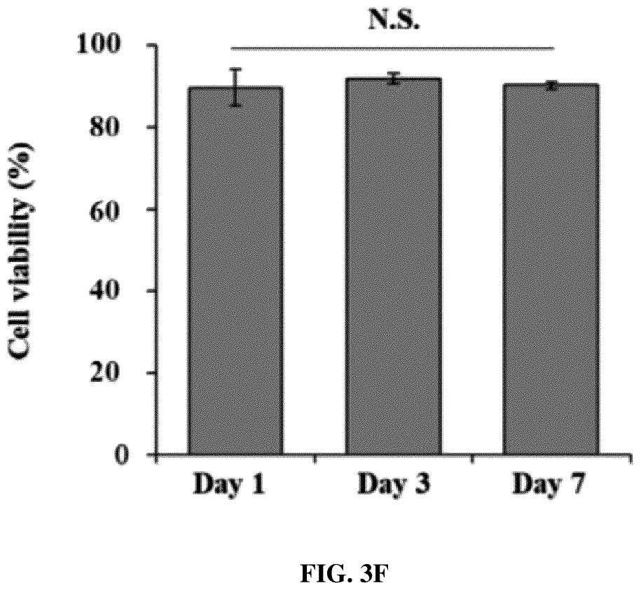

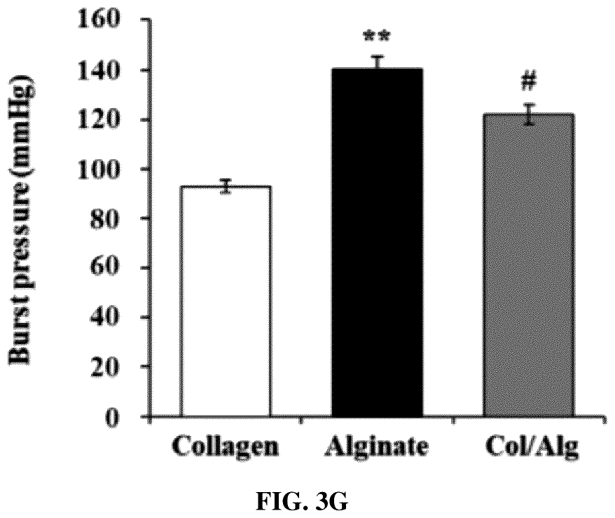

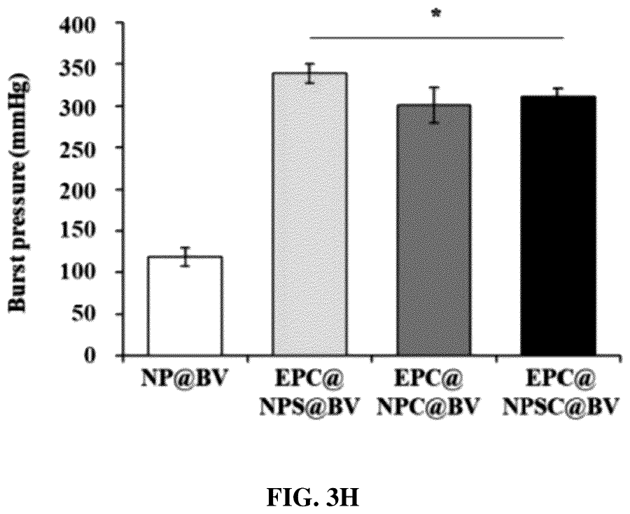

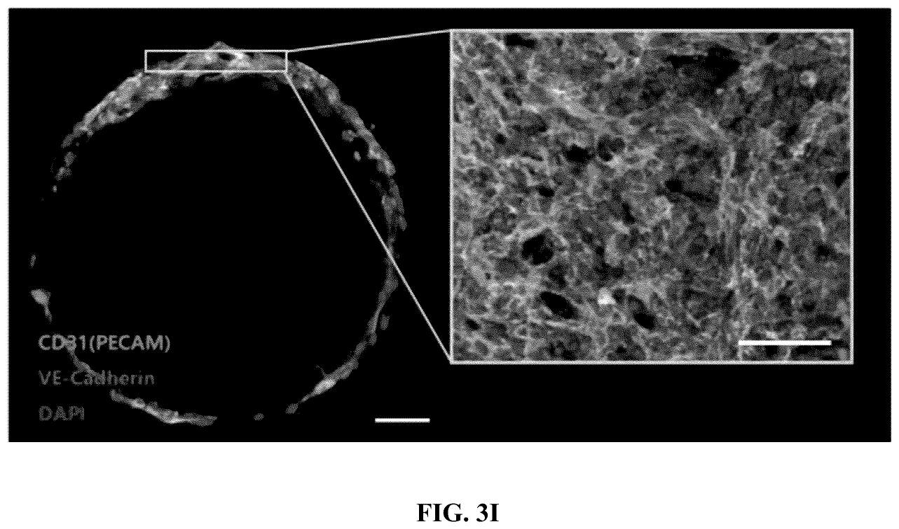

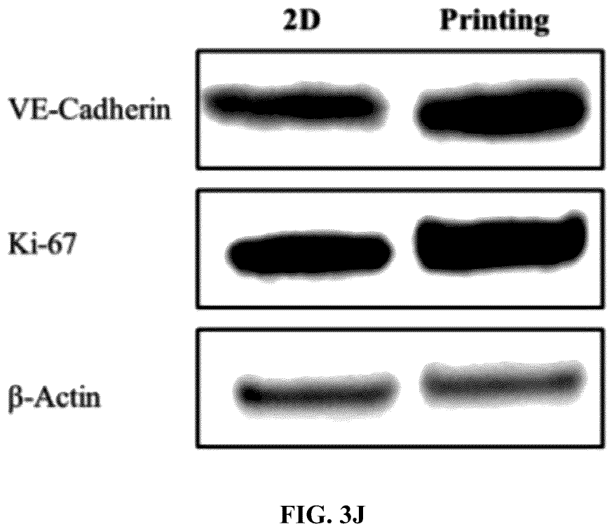

A shell bioink was prepared by mixing 3% wt/v neutralized atelocollagen and 3% wt/v alginate at a 4:1 ratio, and vascular endothelial progenitor cells were mixed with the bioink to have a concentration of 1×10⁶ cells/ml. Then, each of atorvastatin-loaded mesoporous silica (EPC@NPS@BV), curcumin-loaded mesoporous silica (EPC@NPC@BV), atorvastatin-loaded mesoporous silica+curcumin-loaded mesoporous silica (EPC@NPCS@BV), and mesoporous silica (EPC@NP@BV) was mixed with the shell bioink to have a final concentration of 2 mg/ml, thereby preparing artificial blood vessels according to the preparation method of Example 4 (FIGS. 3A and 3B).

Here, artificial blood vessels with various outer diameters (3 mm to 250 μm) and inner diameters (2.5 to 100 μm) were prepared by controlling the thicknesses of the inner and outer needles of the coaxial nozzle of the 3D bioprinter (FIG. 3C), and it was confirmed that stable blood flow could be achieved in the prepared artificial blood vessels (FIG. 3E).

It was confirmed that the vascular endothelial progenitor cells included in the artificial blood vessels could survive stably (FIGS. 3D, 3F, 3I, and 3J), suggesting that the artificial blood vessel of the present disclosure can maintain cell integrity and support tissue regeneration.

In addition, as a result of analyzing the durability of artificial blood vessels made of hydrogels consisting of collagen, sodium alginate, and a collagen/sodium alginate mixture, respectively, it was confirmed that the durability of artificial blood vessels made from sodium alginate was better than that of artificial blood vessels made from collagen, but cell adhesion did not occur when sodium alginate alone was used. Thus, in the present disclosure, a hydrogel prepared by mixing collagen and sodium alginate was used as a bioink (FIG. 3G). In addition, it was confirmed that the optimal durability was achieved when the mixing ratio between collagen and sodium alginate was 2.5:1 to 5:1.

It was confirmed that the durability of the artificial blood vessel including the hydrogel and vascular endothelial progenitor cells was at least three times higher than that of the artificial blood vessel composed of only the hydrogel (FIG. 3H), suggesting that the artificial blood vessel of the present disclosure may be used for vascular replacement in patients.

### Example 1-4. Implantation and Evaluation of Artificial Blood Vessels in Animal Model

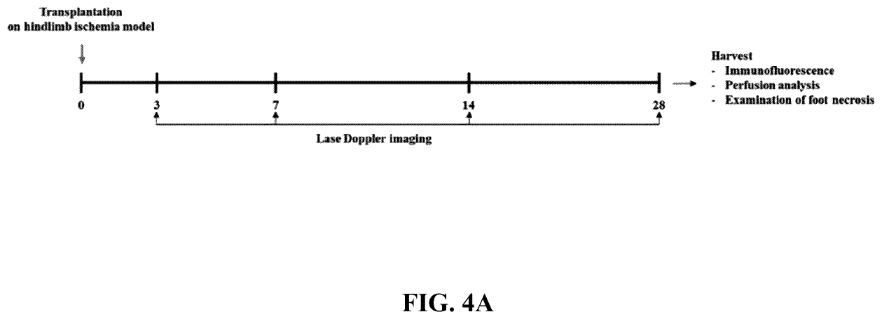

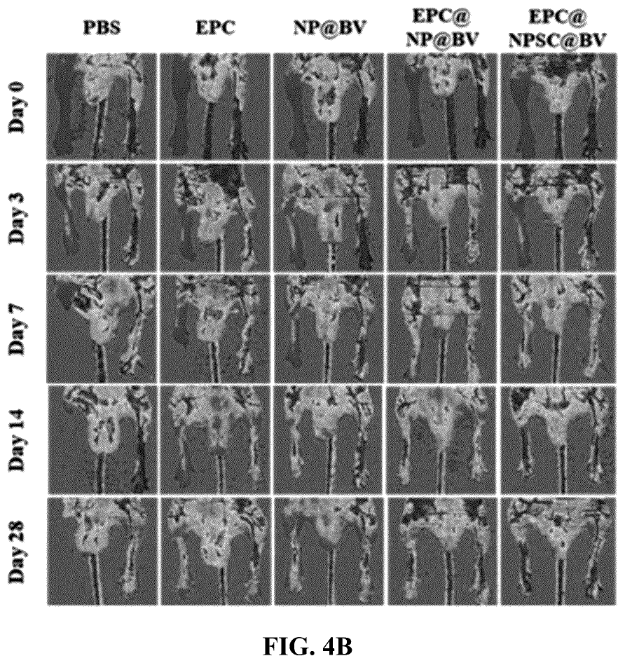

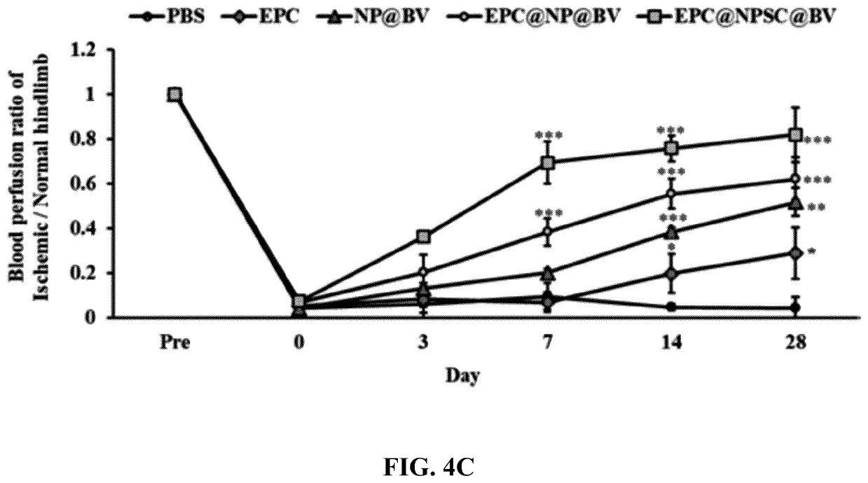

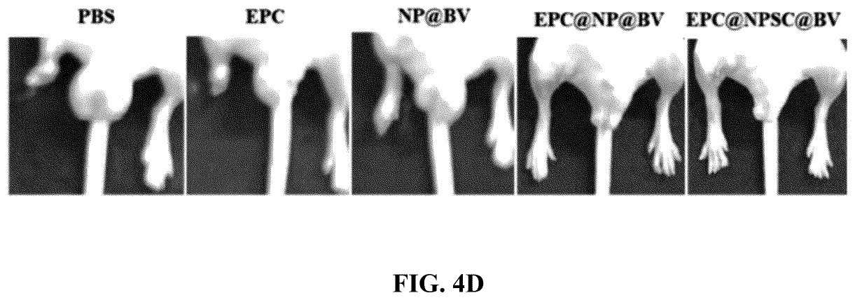

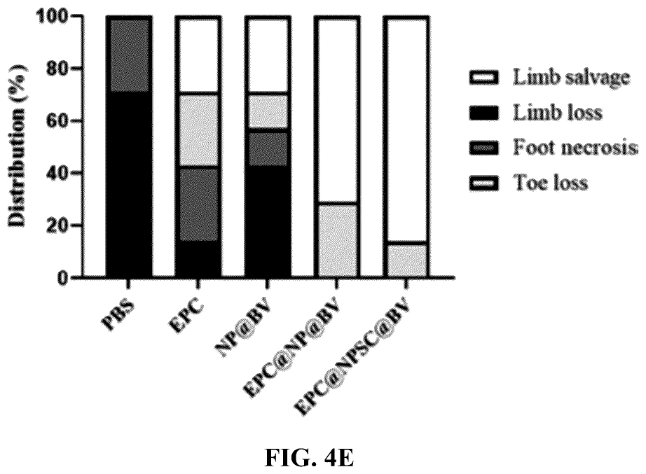

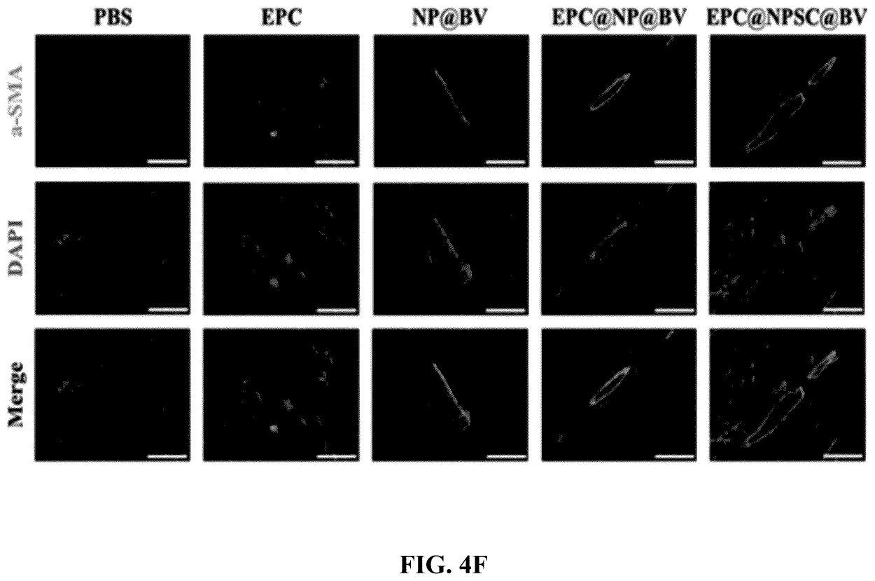

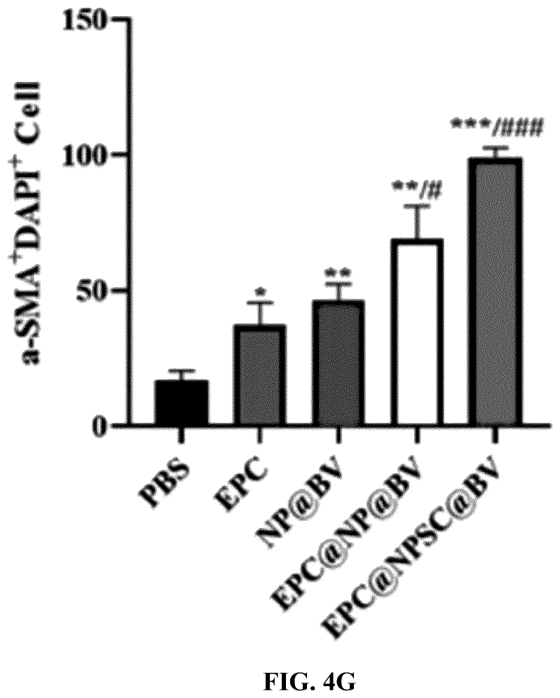

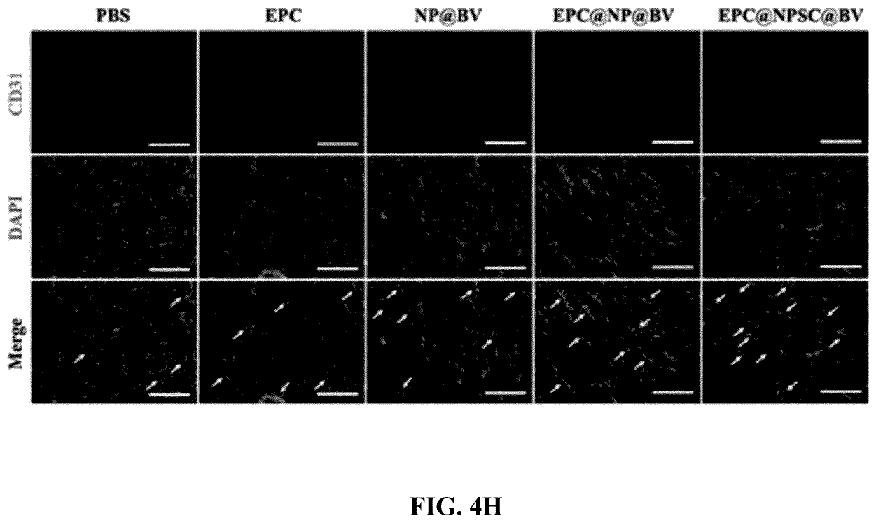

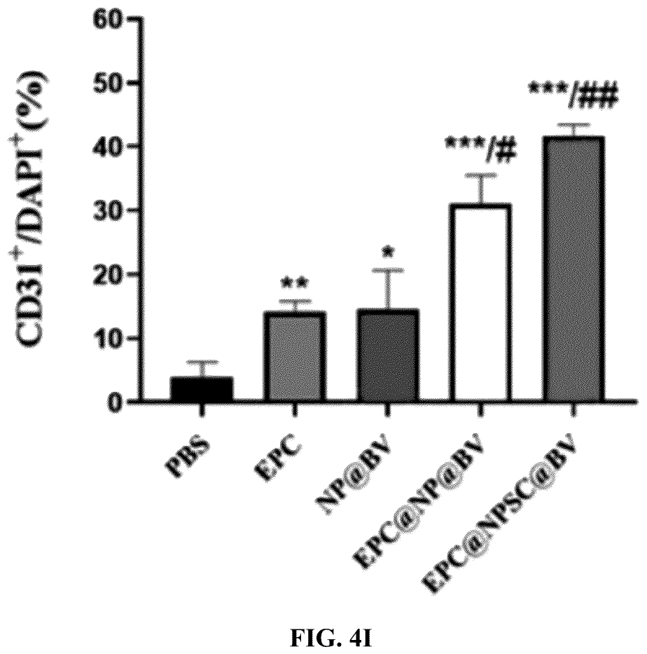

To evaluate the biocompatibility of the previously prepared artificial blood vessels, a mouse hindlimb ischemia model treated with the drugs shown in Table 1 below was prepared using the method of Example 5, and was used to evaluate blood flow and tissue recovery (FIG. 4A).

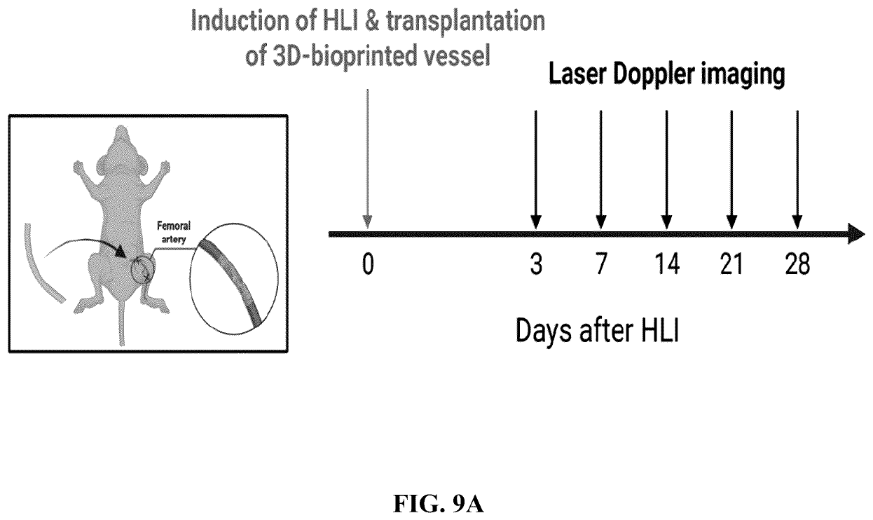

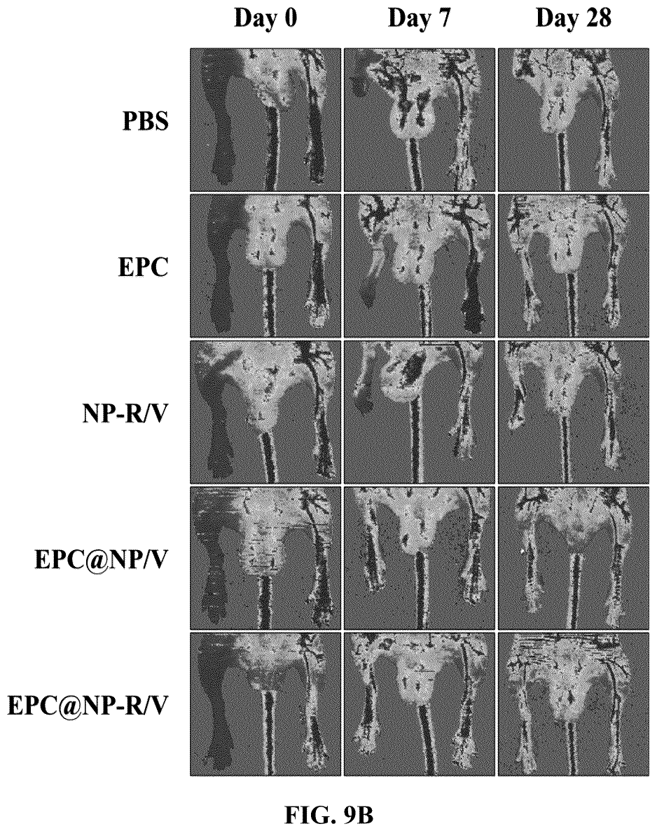

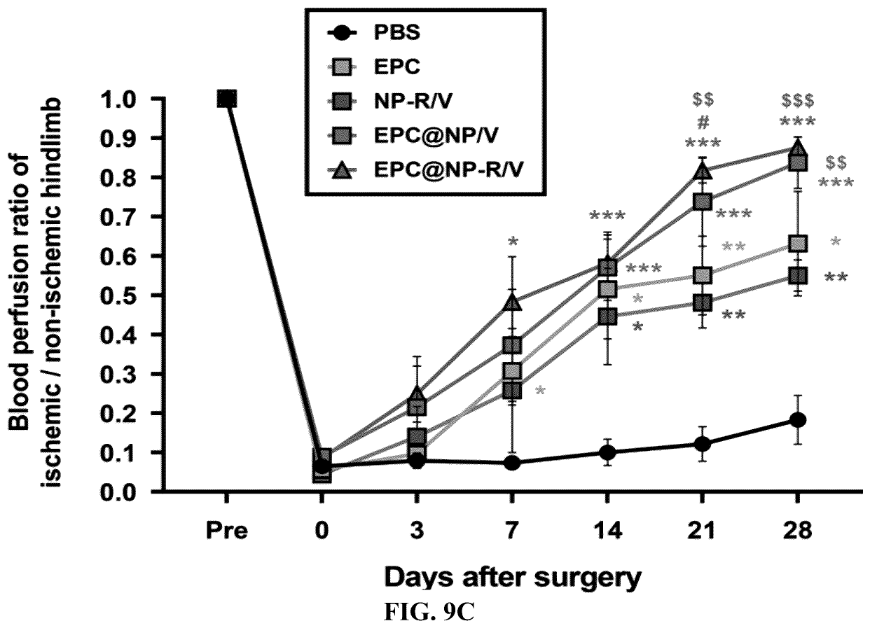

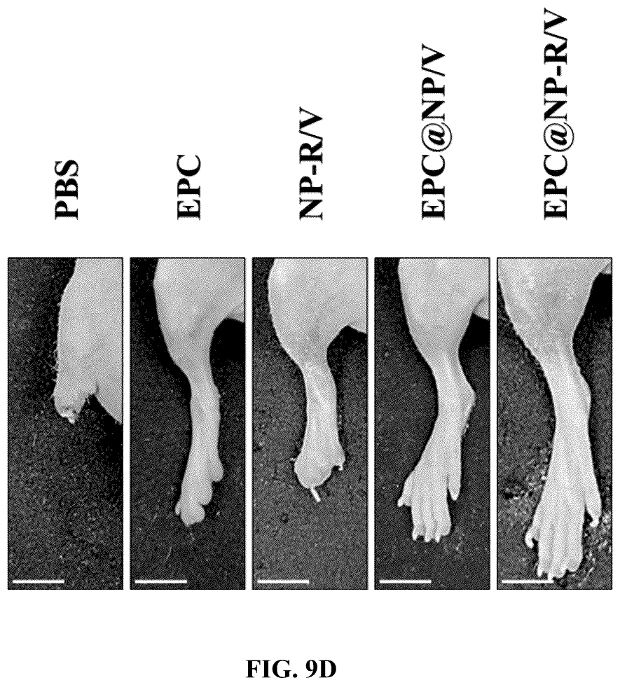

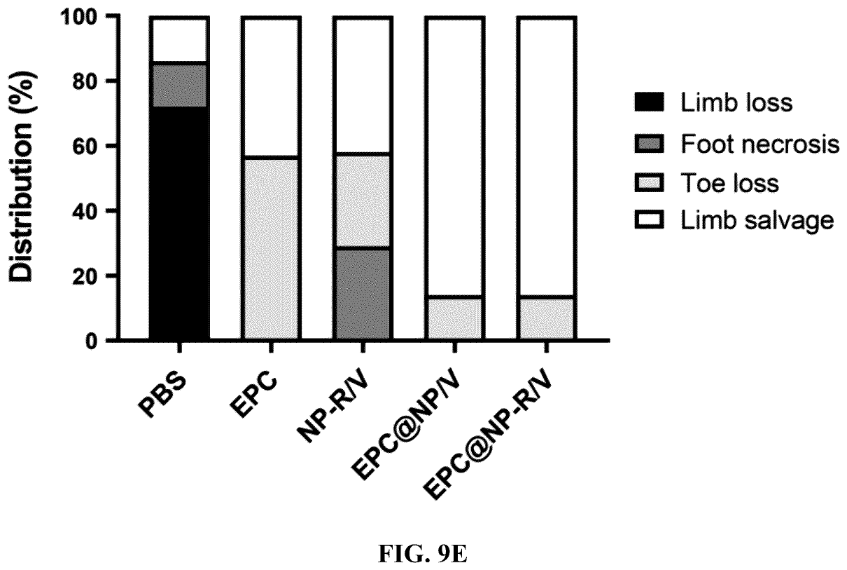

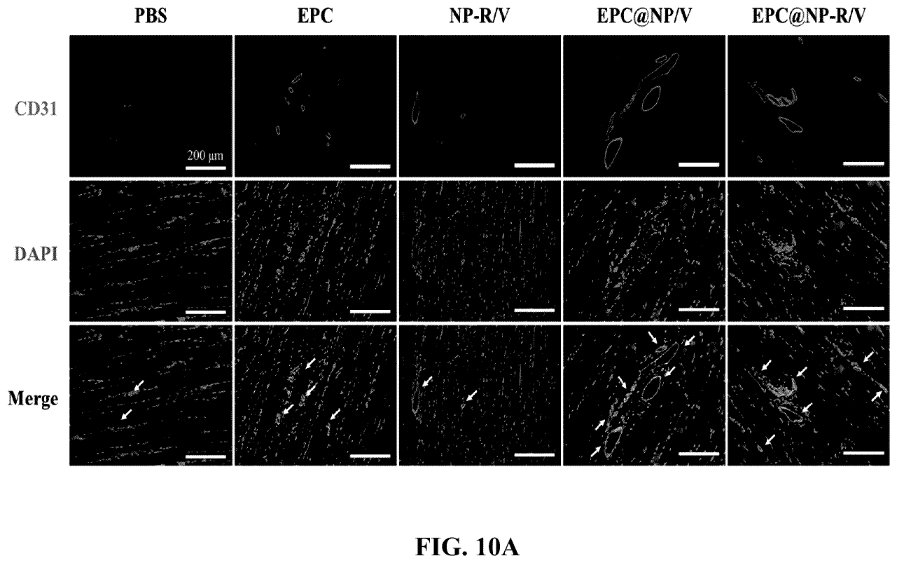

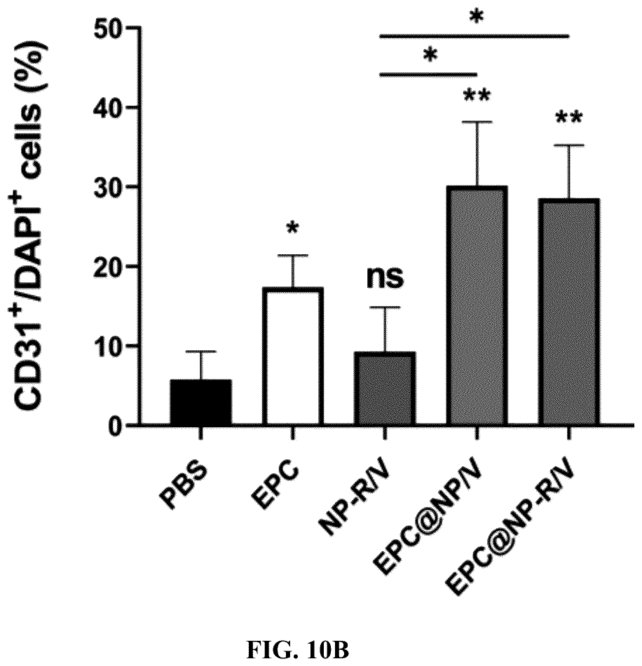

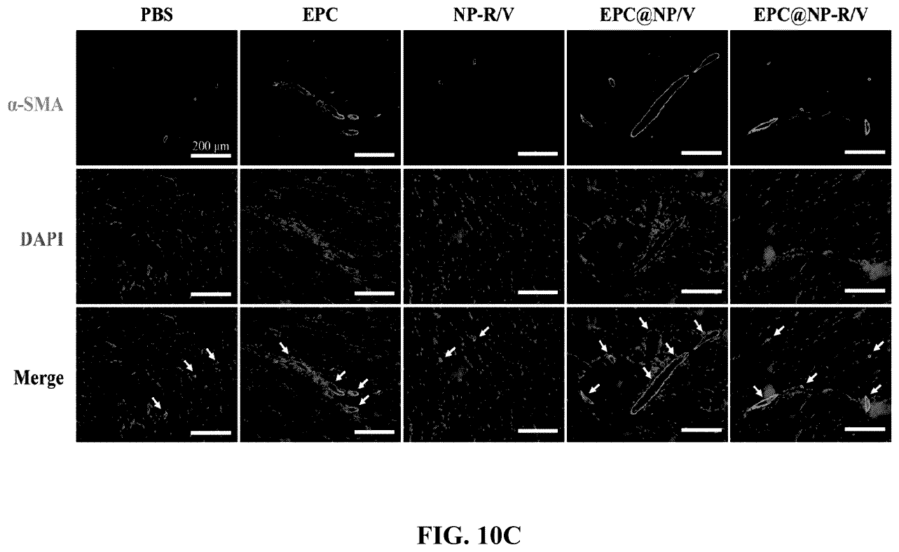

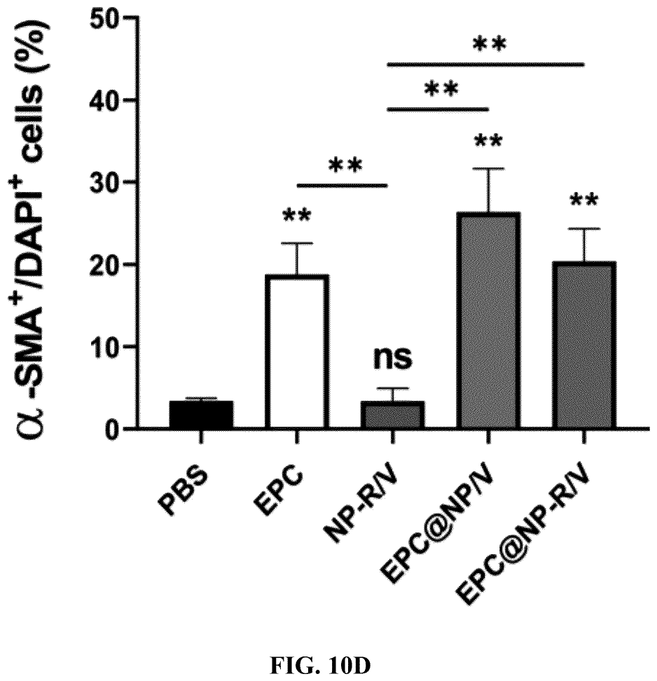

As a result of the experiment, it was confirmed that the PBS-treated group showed complete necrosis on day 7, whereas the artificial blood vessel (EPC@NPSC@BV) group containing both vascular endothelial progenitor cells and drug-loaded mesoporous silica showed blood flow movement from day 3 after surgery and almost complete recovery of blood flow on day 14 (FIGS. 4B to 4E), and also showed an increase in the expression of vascular-related markers (α-SMA and CD31) as indicated by tissue staining (FIGS. 4F to 41). These results show that the artificial blood vessel containing both drug-loaded mesoporous silica and endothelial progenitor cells was successfully implanted to restore blood flow, and that the endothelial progenitor cells present in the artificial blood vessel proliferated, and thus the blood vessel was intrinsically regenerated. This suggests that the artificial blood vessel of the present disclosure can be an effective treatment for ischemic disease.

TABLE 1

| Treatment drug | |
|---|---|
| (symbol) | Detailed description of treatment drug |
| PBS | PBS |
| EPC | Vascular endothelial progenitor cells |
| NP@BV | Artificial blood vessel containing mesoporous |
| | silica (SMB-3) |
| EPC@NP@BV | Artificial blood vessel containing vascular |
| | endothelial progenitor cells and mesoporous |
| | silica (SMB-3) |
| EPC@NPSC@BV | Artificial blood vessel containing vascular |
| | endothelial progenitor cells, curcumin-loaded |
| | mesoporous silica (SMB-3), and atorvastatin- |
| | loaded mesoporous silica (SMB-3) |

### Example 2-1. Production of SR1-Loaded Mesoporous Silica and Patch-Type Scaffold Containing the Same

To produce SR1-loaded mesoporous silica (SMB-3), mesoporous silica (SMB-3) and SR1-loaded mesoporous silica (SMB-3) were produced according to Experimental Example 1, and then a shell bioink was prepared by mixing neutralized atelocollagen (3 w/v %) and sodium alginate (3 w/v %, viscosity >2000 cP) at a ratio of 3:1 according to Experimental Examples 3 and 4. Then, SR1-loaded mesoporous silica (SNP) was mixed with the shell bioink at a final concentration of 1 μM (SNP). A core bioink (sacrificial material) was prepared according to Experimental Example 4, and the shell and core bioinks were printed using a coaxial nozzle (inner needle 19 G and outer needle 14 G) of a 3D bioprinter, thereby fabricating a rod-shaped scaffold.

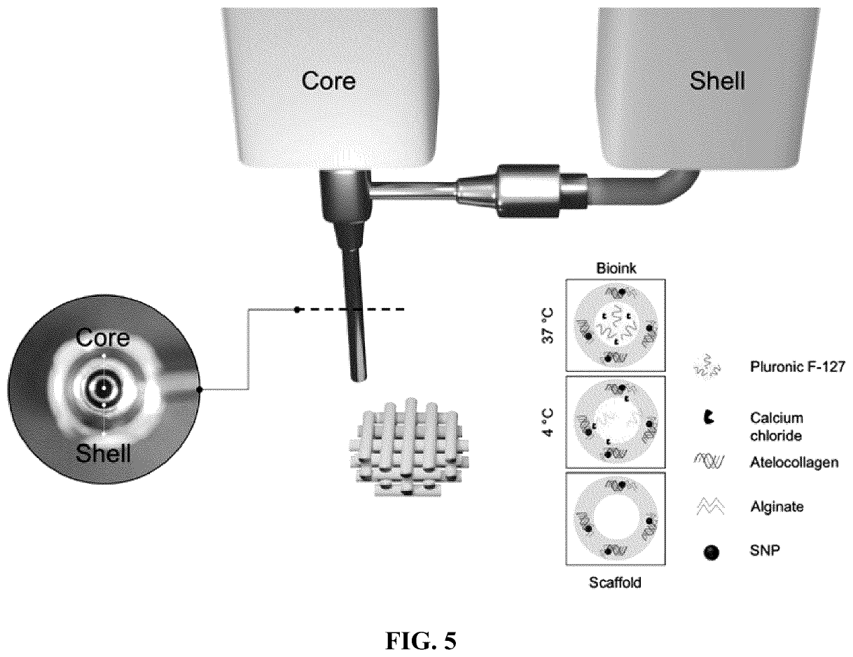

The fabricated rod-shaped scaffold was arranged in a grid pattern (800 mm/min) to make a patch having a size of 40×40×0.6 mm, which was then incubated at 37° C. for one hour to allow the atelocollagen to gel. Next, the patch was trimmed to a diameter of 0.5 mm and then the immersed in PBS at 4° C. to remove the sacrificial material (Pluronic F-127), thereby fabricating a scaffold (FIG. 5A).

### Example 2-2. Implantation and Evaluation of Scaffold in Animal Model

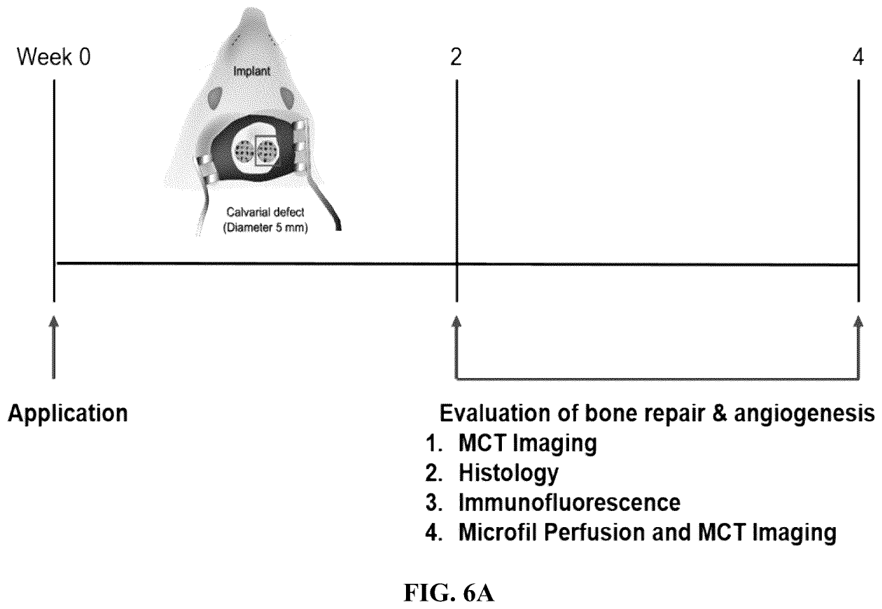

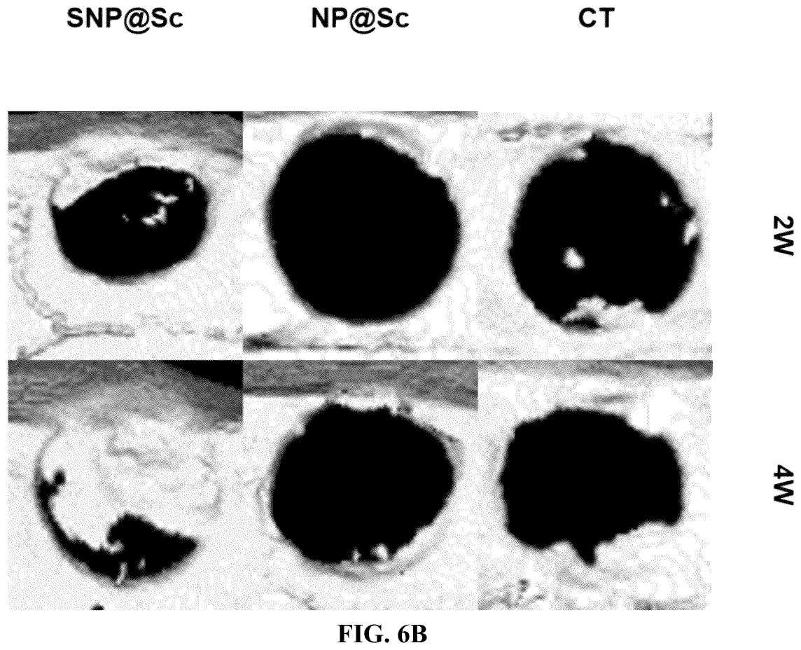

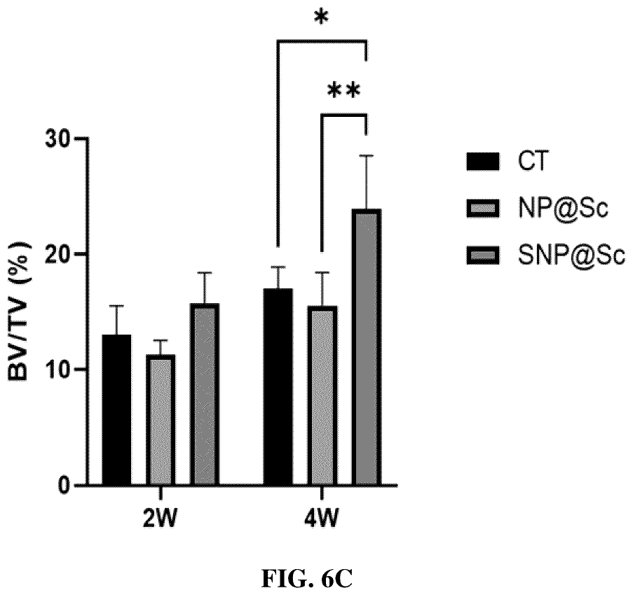

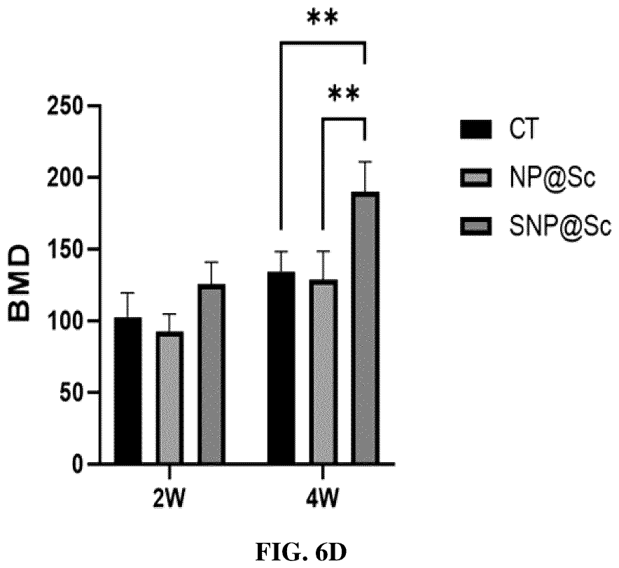

To check whether the scaffold fabricated in Example 2-1 above can actually regenerate bone and blood vessels, a rat calvarial defect model was prepared and the patch-type scaffold shown in Table 2 below was implanted at the calvarial defect site (FIG. 6A).

TABLE 2

| Treatment drug | |
|---|---|
| (symbol) | Detailed description of treatment drug |
| CT | PBS |
| NP@Sc | Scaffold containing mesoporous silica (SMB-3) |
| SNP@Sc | Scaffold containing mesoporous silica (SMB-3) |
| | loaded with SR1 |

FIG. 6B shows MCT images of the defect sites in the rats 2 and 4 weeks after scaffold implantation. Quantitative evaluation of the MCT results is presented in FIG. 6C. The MCT images indicated that new bone was partially regenerated in the defect site, and differences between the groups were observed. As a result of analyzing the bone volume fraction (BV/TV %) of the CT, NP@Sc, and SNP@Sc groups, it was confirmed that the BV/TV values were 13.01±2.51, 11.30±1.23, and 15.75±2.61% area/ROI after 2 weeks, and 17.00±1.87, 15.54±2.83, and 23.91±4.57% area/ROI after 4 weeks, for the CT, NP@Sc, and SNP@Sc groups, respectively. At 2 weeks, the regenerated bone area was in the order of SNP@Sc>CT>NP@Sc. In particular, after 4 weeks, the regenerated bone area in the SNP@Sc group was significantly higher (p<0.01), and this trend persisted in the three groups. In conclusion, these results indicate that SR1 accelerated new bone formation in the rat calvarial defects, with the most notable effects observed at 4 weeks after implantation.

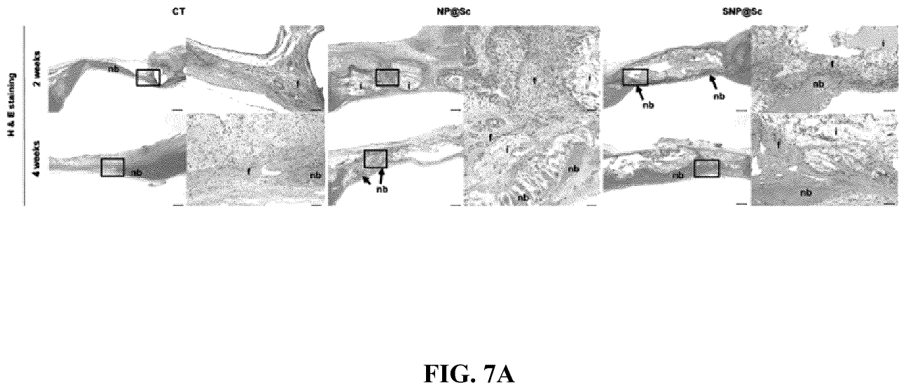

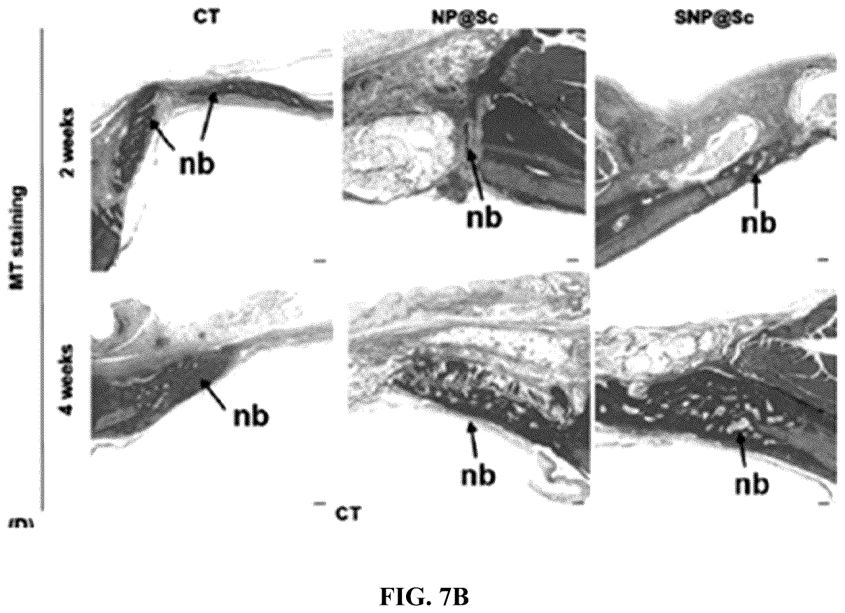

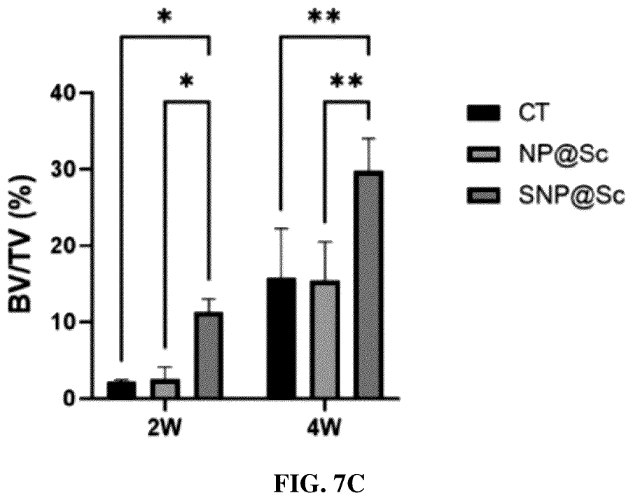

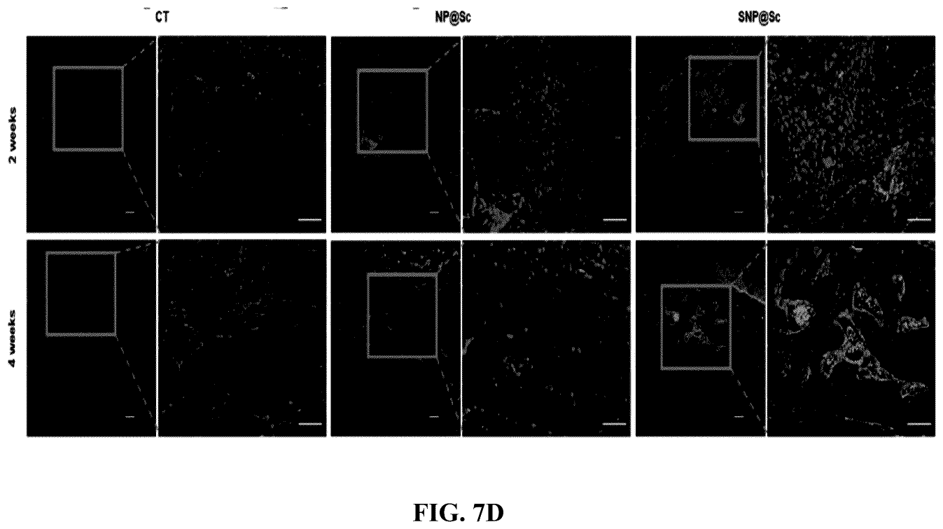

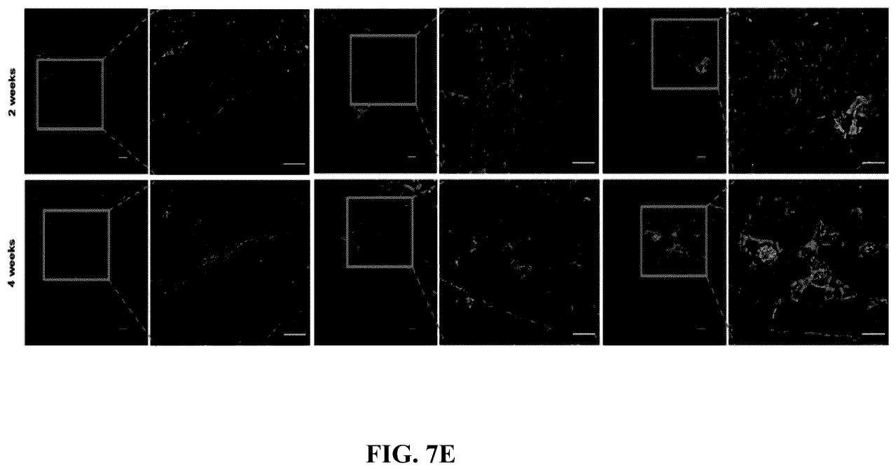

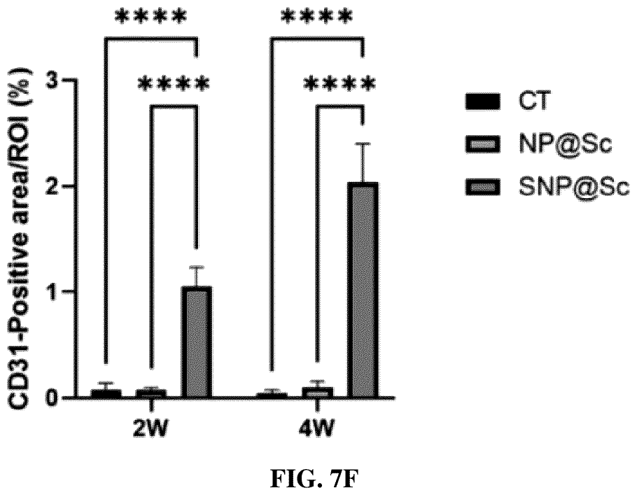

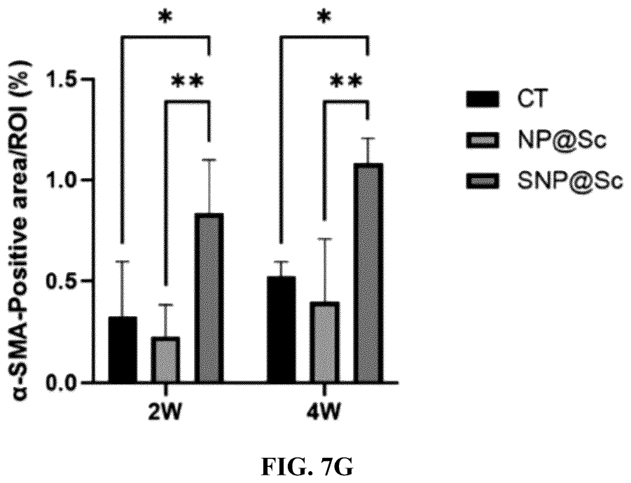

For additional evaluation, histological analysis was performed using H&E and MT staining on sections of rat calvaria after sacrifice. FIGS. 7A and 7B show the H&E and MT staining results of the rat calvarial samples obtained at 2 and 4 weeks after scaffold implantation. FIG. 7C illustrates that new bone formation in the SNP@Sc group was significantly higher than that in the CT and NP@Sc groups 2 weeks and 4 weeks after implantation. In addition, the areas of the newly formed bone were 2.14±0.24, 2.51±1.60, 11.29±1.72 mm² after 2 weeks, and 15.81±6.39, 15.40±5.10, 29.75±4.25 mm² after 4 weeks, in the CT, NP@Sc, and SNP@Sc groups.

Immunofluorescence staining was performed on the paraffin-sectioned slides to analyze CD31 and α-SMA expression in the newly formed bone areas (FIGS. 7D and 7E). These results were confirmed using the MT-stained slides (FIG. 7B). In addition, FIGS. 7F and 7G show the quantitative indices of CD31-positive and α-SMA-positive sections. The expression levels of CD31 and α-SMA exhibited no significant difference between the CT and NP@Sc groups, but the SNP@Sc group displayed significantly higher expression levels of CD31 and α-SMA. The expression levels of CD31 were 0.07±0.07, 0.07±0.03, and 1.06±0.18% area/ROI after 2 weeks, and 0.05±0.03, 0.10±0.06, 2.04±0.36% area/ROI after 4 weeks, in the CT, NP@Sc, and SNP@Sc groups, respectively (p<0.0001). The expression levels of α-SMA were 0.33±0.27, 0.23±0.15, 0.83±0.26% area/ROI after 2 weeks, and 0.53±0.08, 0.40±0.31, 1.08±0.12% area/ROI after 4 weeks, in the CT, NP@Sc, and SNP@Sc groups, respectively (p<0.05 when compared to CT; p<0.01 when compared to NP@Sc). The IF staining results indicated the enhanced regeneration of blood vessels in the SNP@Sc group compared to the other groups.

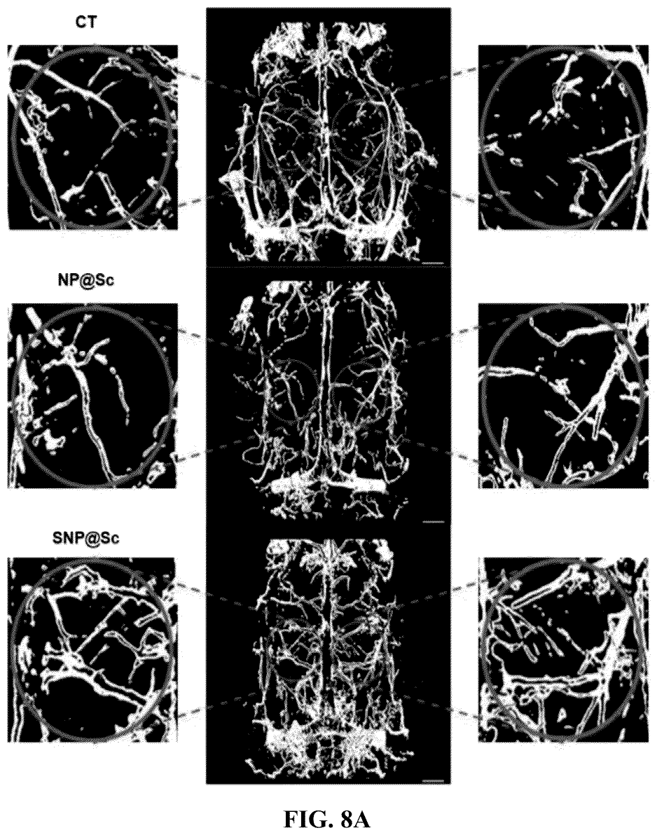

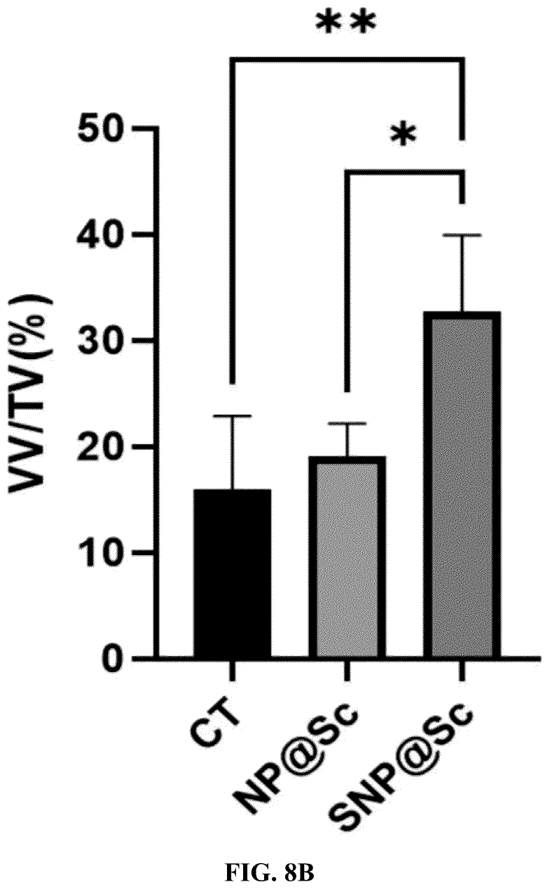

Images were obtained using the MCT system after cardiac perfusion with a Microfil compound to observe the vasculature at the implantation site. FIG. 8A shows the implantation site and cranial vessels. More capillaries were observed in the SNP@Sc group than in the other groups. However, there was no significant difference in the number of blood vessels formed between the CT and NP@Sc groups. Quantitative analysis was performed using MCT images for all groups (FIG. 8B). The vascular volume/total volume (VV/TV %) was analyzed, giving values of 15.94±4.62%, 19.05±2.64%, and 32.67±6.51% at 4 weeks after implantation in the CT, NP@Sc, and SNP@Sc groups, respectively. A significantly higher volume of the vascular network was observed in the SNP@Sc group than in the other groups (p<0.01 when compared to CT; p<0.05 when compared to NP@Sc). The results of analysis of vascular formation suggest that SR1 treatment promoted neovascularization in the rat calvarial defects.

Taking the above results together, it can be seen that the scaffold for bone formation according to the present disclosure can successfully regenerate defective bone tissue and blood vessels using cells existing around the surgical site, even though the scaffold does not contain progenitor cells for tissue regeneration within the scaffold.

Although the preferred embodiments of the present disclosure have been described in detail above, the scope of the present disclosure is not limited thereto, and various modifications and improvements can be made by those skilled in the art without departing from the basic concept of the present disclosure as defined in the following claims, and also fall within the scope of the present disclosure.

What is claimed is:

1. A biocompatible hydrogel composition comprising: a spherical mesoporous silica loaded with a drug; sodium alginate; and collagen,
wherein the mesoporous silica is capable of releasing the loaded drug in a controlled and sustained manner.

2. The biocompatible hydrogel composition according to claim 1, wherein an inside of the mesoporous silica includes a plurality of mesopores, and a surface of the mesoporous silica has fine grooves derived from the mesopores.

3. The biocompatible hydrogel composition according to claim 1, wherein the mesopores in the mesoporous silica have incorporated metal nanoparticles composed of at least one of zinc, zinc oxide, silver, platinum, gold, and mixtures thereof.

4. The biocompatible hydrogel composition according to claim 1, wherein the drug is selected from the group consisting of small-molecule substances, antibodies, antibiotics, immunosuppressants, anticancer agents, vascular narrowing inhibitors, cell growth promoters, and combinations thereof.

5. The biocompatible hydrogel composition according to claim 1, wherein a mixing ratio between the collagen and the sodium alginate is 2.5:1 to 5:1.

6. The biocompatible hydrogel composition according to claim 1, further comprising mesenchymal stem cells or tissue progenitor cells.

7. A bioink comprising the biocompatible hydrogel composition according to claim 1.

8. An artificial blood vessel made from the bioink according to claim 7.

9. A scaffold for bone regeneration made from the bioink according to claim 7.
